SEQUENCE LISTING (74 sequences)

SEQ ID NO: 1 - protein, 13 aa, Borreliella afzelii
ININYAQEDDINK

SEQ ID NO: 2 - protein, 11 aa, Borrelia parkeri
FEINLDYVNEK

SEQ ID NO: 3 - protein, 14 aa, Borrelia turicatae
MLLADEKDVIDSIR

SEQ ID NO: 4 - protein, 19 aa, Borrelia duttonii
GGAQIIDPHDAKIISEIQK

SEQ ID NO: 5 - protein, 19 aa, Borrelia hermsii
MGLAQPVITQQMVIAELTR

SEQ ID NO: 6 - protein, 27 aa, Borreliella burgdorferi
YFDNLSEEDVLKLNIPTGIPLVYELDK

SEQ ID NO: 7 - protein, 19 aa, Borreliella burgdorferi
LGEVVAFFEYLNSVFLEVR

SEQ ID NO: 8 - protein, 10 aa, Borrelia crocidurae
MITSIIEINK

SEQ ID NO: 9 - protein, 13 aa, Borrelia hermsii
DTDIFYYYRLDNK

SEQ ID NO: 10 - protein, 9 aa, Borrelia turicatae
GLGGIANIK

SEQ ID NO: 11 - protein, 22 aa, Borrelia crocidurae
GGNFVYSTLEFQVMQEDIEICK

SEQ ID NO: 12 - protein, 11 aa, Borrelia turicatae
VINLNIEAGHK

SEQ ID NO: 13 - protein, 11 aa, Borrelia duttonii
NVVNAAYEVIK

SEQ ID NO: 14 - protein, 22 aa, Borrelia turicatae
RNVDNFDLSHLNWLWNFDYAGK

SEQ ID NO: 15 - protein, 10 aa, Borrelia persica
SVFMLAGEIK

SEQ ID NO: 16 - protein, 10 aa, Borrelia turicatae
GLGSFDLQDK

SEQ ID NO: 17 - protein, 14 aa, Borrelia crocidurae
NMLDIISNNLNIIR

SEQ ID NO: 18 - protein, 11 aa, Borrelia turicatae
LDDIPGIIHSK

SEQ ID NO: 19 - protein, 15 aa, Borrelia parkeri
AVEESVVAMQSIVEK

SEQ ID NO: 20 - protein, 36 aa, Borrelia duttonii
NGVDGPLTKPLSPGGPQGVTNPGNGGGAVNDEQILK

SEQ ID NO: 21 - protein, 18 aa, Borrelia duttonii
KMIAEYETLQQQIFKMQR

SEQ ID NO: 22 - protein, 21 aa, Borrelia persica
RTDIASFQANIDITIDSLNSR

SEQ ID NO: 23 - protein, 25 aa, Borreliella bavariensis
SALTVGVGTIMDSQEILIIVNGHNK

SEQ ID NO: 24 - protein, 11 aa, Borrelia duttonii
FLSQQKDLEDK

SEQ ID NO: 25 - protein, 12 aa, Borrelia turicatae
INLYLVEGIDNK

SEQ ID NO: 26 - protein, 25 aa, Borrelia hermsii
ISAIIMTLFMVLVSCNSGGVAEDPK

SEQ ID NO: 27 - protein, 17 aa, Borrelia crocidurae
MDEMKSSFGILSNLLMK

SEQ ID NO: 28 - protein, 20 aa, Borrelia crocidurae
NLYNTFLNIGTYFQMIDDIK

SEQ ID NO: 29 - protein, 16 aa, Borreliella burgdorferiss
LENTAMPLVAEIGEVK

SEQ ID NO: 30 - protein, 39 aa, Borrelia parkeri
EIIPTINGELLDNIVDYINYTFIPTIFFYYGNFISNEYK

SEQ ID NO: 31 - protein, 21 aa, Borreliella bavariensis
IVTHNDLNGKNNSNSSINLDK

SEQ ID NO: 32 - protein, 18 aa, Borrelia turicatae
NPIINEQQTLLTSIAWGR

SEQ ID NO: 33 - protein, 14 aa, Borrelia duttonii
FAEFLLDTLFLNNK

SEQ ID NO: 34 - protein, 10 aa, Borrelia duttonii
LNENELFDVK

SEQ ID NO: 35 - protein, 12 aa, Borrelia duttonii
NEVTSITEDEQK

SEQ ID NO: 36 - protein, 13 aa, Borrelia recurrentis
LPEVLYAYELDIR

SEQ ID NO: 37 - protein, 17 aa, Borrelia duttonii
ENENETFQSEDNPNMEK

SEQ ID NO: 38 - protein, 20 aa, Borrelia turicatae
VETNGSSGVDNLIEEEEIER

SEQ ID NO: 39 - protein, 20 aa, Borrelia crocidurae
NLILINTNEFHNGIVLNDPR

SEQ ID NO: 40 - protein, 12 aa, Borrelia parkeri
NADISYYNQGIK

SEQ ID NO: 41 - protein, 17 aa, Borrelia hermsii
GVTELEALNTAVDALLK

SEQ ID NO: 42 - protein, 16 aa, Borrelia parkeri
EKLLDEQIIQELSAIK

SEQ ID NO: 43 - protein, 18 aa, Borrelia crocidurae
IFLSENVSILNESLNILR

SEQ ID NO: 44 - protein, 19 aa, Borrelia hermsii
QILAEFVDNNQLGYSINNR

SEQ ID NO: 45 - protein, 22 aa, Borreliella burgdorferiss
INTYDIPQNNNLETEDVNEPNK

SEQ ID NO: 46 - protein, 26 aa, Borrelia duttonii
LVRDSLLFYSSTNLAILNFSIANNIK

SEQ ID NO: 47 - protein, 12 aa, Borrelia hispanica
SIVGTMIDIEIK

SEQ ID NO: 48 - protein, 9 aa, Borrelia turicatae
YAELLEINK

SEQ ID NO: 49 - protein, 11 aa, Borreliella afzelii
KDFNNNEIELR

SEQ ID NO: 50 - protein, 14 aa, Borrelia turicatae
SLNIDITGDKVELR

SEQ ID NO: 51 - protein, 19 aa, Borreliella burgdorferiss
IVDIDESNPNLPYINYIIK

SEQ ID NO: 52 - protein, 14 aa, Borrelia turicatae
LFMSELSSIELSER

SEQ ID NO: 53 - protein, 19 aa, Borrelia turicatae
LIGLQTEPNVIEFNDFLNK

SEQ ID NO: 54 - protein, 20 aa, Borrelia turicatae
TNPNEINALQTLNDYYYSNK

SEQ ID NO: 55 - protein, 10 aa, Borrelia sp.
IILGASGAEK

SEQ ID NO: 56 - protein, 13 aa, Borreliella burgdorferiss
NYLNEPSRDIITK

SEQ ID NO: 57 - protein, 11 aa, Borreliella burgdorferiss
SLEVLGNEYTK

SEQ ID NO: 58 - protein, 15 aa, Borrelia crocidurae
ESISEQDIMQSSLNK

SEQ ID NO: 59 - protein, 16 aa, Borrelia persica
ENPNVLQIMQVEEDIK

SEQ ID NO: 60 - protein, 13 aa, Borrelia duttonii
LGEASEELEKVAK

SEQ ID NO: 61 - protein, 15 aa, Borreliella afzelii
LNCNLVFIDGKFVPK

SEQ ID NO: 62 - protein, 18 aa, Borrelia hermsii
DLLIVGLGNPGSNFFHTR

SEQ ID NO: 63 - protein, 16 aa, Borrelia turicatae
QDNLNFLSLEDLINLK

SEQ ID NO: 64 - protein, 20 aa, Borrelia turicatae
MFLVMFFVSCVLFSEEIYYR

SEQ ID NO: 65 - protein, 10 aa, Borrelia hermsii
SGDIDLEVLK

SEQ ID NO: 66 - protein, 22 aa, Borreliella afzelii
ETGHENAYFPMLIPYGFLEKEK

SEQ ID NO: 67 - protein, 11 aa, Borrelia crocidurae
ASEELEEVAKK

SEQ ID NO: 68 - protein, 12 aa, Borrelia duttonii
SGYIFEGINGVF

SEQ ID NO: 69 - protein, 22 aa, Borrelia recurrentis
DKEFISQMAQFSALEQMTNMNK

SEQ ID NO: 70 - protein, 9 aa, Borrelia miyamotoi
LSEIFEMIK

SEQ ID NO: 71 - protein, 22 aa, Borrelia crocidurae
IQGWMAESVGGEQVFNTSSDIR

SEQ ID NO: 72 - protein, 21 aa, Borreliella burgdorferiss
ISAALSDTGVTYSRFIEGLLK

SEQ ID NO: 73 - protein, 26 aa, Borrelia turicatae
TISYLNNIFNNEILRQEIMSTNDINR

SEQ ID NO: 74 - protein, 13 aa, Borrelia parkeri
EIECAVIGNEQIK

(19) United States
(12) Patent Application Publication
(10) Pub. No.: US 2025/0164482 A1
(43) Pub. Date: May 22, 2025

(54) AFFINITY FILAMENTOUS MATERIAL FOR LYME DISEASE BIOMARKER CAPTURE FROM BIOLOGICAL SAMPLES

(71) Applicant: GEORGE MASON UNIVERSITY, Fairfax, VA (US)

(72) Inventors: Alessandra Luchini KUNKEL, Fairfex, VA (US); Lance Allen LIOTTA, Fairfex, VA (US); Barbara BIRKAYA, Fairfex, VA (US); Rocío Solange PRISBY, Fairfex, VA (US); Sumaiya Safia IRFAN, Fairfex, VA (US)

(21) Appl. No.: 18/953,424

(22) Filed: Nov. 20, 2024

Related U.S. Application Data
(60) Provisional application No. 63/600,994, filed on Nov. 20, 2023.

(51) Int. Cl. G01N 33/569; G01N 33/68

(52) U.S. Cl. CPC G01N 33/56911; G01N 33/6848; G01N 2333/20

(57) ABSTRACT

An embodiment of the invention relates to a method for diagnosing Lyme disease, comprising: isolating a diagnostic composition from a biological sample, wherein the biological sample is urine or another bodily fluid, the diagnostic composition comprising a biomarker attributable to Borrelia strains, bound or complexed to an affinity molecule that is bound or complexed to a filamentous material; and analyzing isolated diagnostic composition using a mass spectrometry, an immunoassay, a protein sequencing, or other analytical methods to detect presence of Borrelia-derived biomarkers.

### RELATED APPLICATION

This application claims priority to U.S. provisional application No. 63/600,994, filed on Nov. 20, 2023, titled as “IDENTIFICATION OF INFECTIOUS PEPTIDES IN HUMAN URINE” which is herein incorporated by reference in its entirety.

This application relates to U.S. Ser. No. 17/622,403, titled, “Diagnostic method for infectious diseases” which is herein incorporated by reference in its entirety.

### FIELD OF THE INVENTION

This invention relates to a process of identification of biomarkers present in biofluids. The invention is more particularly concerned with a process involving identification of the biomarkers of Borreliosis in a biofluid.

### SEQUENCE LISTING

The instant application contains a Sequence Listing submitted electronically in XML titled GMUN-036-01US.xml created on Oct. 14, 2024, using WIPO ST.26 sequence listing. The said XML file is 63.9 KB in size hereby incorporated by reference in its entirety.

### BACKGROUND OF INVENTION

Diagnosing diseases using biomarkers contained in biofluids, such as blood and urine, can provide information on the health of entire organism containing the biofluids.

Biofluids are extremely important because of their non-invasive nature and because they integrate biomarker abundance data over time. For example, circulating biomarkers, which may be intermittently released in the blood or other fluid, are concentrated and integrated, and eventually, secreted. Particularly, peptides/proteins are emphasized because they can provide unique insights, due to their functional role, in mediating disease phenotype1. Disease-related peptides/proteins carry out ongoing pathological functions, including adaptation to therapies, tissue-specific host interactions, immune evasion, and tissue tropism.

Diagnosing diseases based on biological samples can be challenging; while providing valuable patient insights, it may not always be sufficient for a definitive diagnosis. The exceedingly low concentration of disease-derived proteins in, for example, urine, and the genetic diversity that some pathogens can present² pose an extraordinary analytical challenge for robust detection. Similarly, rare proteins in the blood and urine matrix are masked by high abundance resident proteins, such as uromodulin and albumin, that can evade disease identification¹. Diagnosing diseases using biomarkers, contained in biofluids, can also be challenging since the biomarkers may not always be sufficient, sensitive, or specific enough for a definitive diagnosis.

Conventional methods such as enzyme-linked immunosorbent assays (ELISA), polymerase chain reaction (PCR), and immunohistochemistry (IHC) can present limited specificity, sensitivity or cross-reactivity, longer turnaround times, variability in diagnosis due to pre-analytical factors like sample handling, storage conditions, freeze-thaw cycles, and variability in performance cost and complexity.

Examples of previous patent literature on biomarker capture/discovery: Smart hydrogel particles for biomarker harvesting (U.S. Pat. No. 7,935,518B2), Method for harvesting nanoparticles and sequestering biomarkers (U.S. Pat. No. 8,382,987B2), Hydrogel nanoparticle based immunoassay (U.S. Pat. No. 9,012,240B2), Smart Hydrogel Particles for Biomarker Harvesting (US20120164749A1), Borrelia burgdorferi bacterial antigen diagnostic test using polymeric bait containing capture particles (US20130085076A1), Diagnostic and therapeutic methods for cancer (U.S. Pat. No. 11,473,151B2).

Therefore, there is a need for a low-cost, time efficient test to capture and analyze these biomarkers for disease diagnostics.

### SUMMARY OF INVENTION

The present disclosure relates to innovative compositions and methodology for diagnosing Lyme disease through the analysis of urine and other bodily fluids. Specifically, the disclosure provides for the direct identification and sequencing of peptides derived from pathogenic strains and species of Borrelia.

An embodiment relates to a method, comprising: taking a filamentous material functionalized with one or more affinity agents specific to one or more biomarkers, wherein a weight ratio (% W/W) between a total amount of affinity agents attached to the filamentous material is about 0.5 to 2%; contacting the filamentous material with a volume of a biological fluid to allow affinity agents to capture biomarkers present in the biological fluid in a suitable condition; eluting captured biomarkers; and wherein the said method is a non-invasive process.

In an embodiment, the filamentous material functionalized with one or more affinity agents is a non-imbibing material.

In an embodiment, the captured biomarkers are analyzed.

In an embodiment, analysis of the captured biomarkers comprises matching a chemical composition of the captured biomarkers with a known database.

In an embodiment, the method is configured to detect a disease in a subject.

In an embodiment, the filamentous material comprises a polymer comprising nylon-6, and/or polyaminde.

In an embodiment, the method is configured to concentrate one or more biomarkers present in the biological fluid sample by at least 1000-fold.

In an embodiment, the method has sensitivity of about 90% to detect a target biomolecule in the biological fluid sample.

In an embodiment, the method has specificity of about 95% to detect a target biomolecule in the biological fluid sample.

In an embodiment, the method has specificity to detect a target biomolecule present at a concentration of about 2.5 picograms/mL or less in the biological fluid sample.

In an embodiment, the suitable condition comprises pH of the biological fluid.

An embodiment relates to a method, comprising: taking a filamentous material functionalized with one or more affinity agents specific to one or more biomarkers, wherein a weight ratio (% W/W) between a total amount of affinity agents attached to the filamentous material is about 0.5 to 2%; contacting the filamentous material with a volume of a biological fluid to allow affinity agents to capture biomarkers present in the biological fluid in a suitable condition; eluting captured biomarkers; and analyzing the captured biomarkers; wherein the method is configured to detect a tick-borne pathogen in the biological fluid.

In an embodiment, the tick-borne pathogen comprises Borellia asps.

In an embodiment, the biological fluid comprises urine.

In an embodiment, the biological fluid is not preserved in a refrigerated condition before contacting the filamentous material.

In an embodiment, the method is configured to detect peptides related to Borrelia sps. at a concentration of 2.5 picograms/ml in the biological fluid.

In an embodiment, the filamentous material comprises a non-imbibing material.

In an embodiment, pH of urine is about 5.5 before contacting the filamentous material.

In an embodiment, the filamentous material comprises a polymer.

In an embodiment, the polymer is heated at a temperature about its glass transition temperature to allow functionalization with one or more affinity agents.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying drawings are included to provide further understanding of the present invention disclosed in the present disclosure and are incorporated in and constitute a part of this specification, illustrate aspects of the present invention and together with the description serve to explain the principles of the present invention. In the drawings:

FIG. 1 shows an outline of the experimental procedure to identify disease-specific peptides in biofluids. Biofluids are incubated with an affinity net, digested, and subjected to mass spectrometry analysis. A bioinformatics pipeline ensures peptide specificity.

FIG. 2 shows stained thread (left) and affinity net (right) obtained with dye Sudan Black B (A) and Sudan IV (B).

FIG. 3 shows heterocyclic rings comprising thiazoles, thiazones, thianthrenes, and phenothiazonethioanthrone.

FIG. 4 shows an example of a reactive dye.

FIG. 5 shows phthalocyanine dyes comprising tetraazo or a derivative of tetrabenzoporphyrin consisting of four isoindole units connected by four nitrogen atoms.

FIG. 6 shows examples of acid dyes.

FIG. 7 shows SDS PAGE analysis of a typical successful protein capture and elution from the affinity net. Lane 1: ladder, Lanes 2-4: urine initial solution (Input), Lanes 5-7: supernatant (left behind after affinity net incubation), Lanes 8-10: eluates (proteins eluted from the affinity net).

FIG. 8 shows a concept to establish and verify a urine protein biomarker-based test for the direct diagnosis of Borreliosis.

FIG. 9A shows outer surface protein C Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9B shows flagellar motor switch protein FliN Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9C shows chain A 2, 3 biphosphoglycerate-dependent phosphoglycerate mutase Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9D shows flagellar motor switch protein FliG Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9E shows hypothetical protein BT0238 Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9F shows flagellin Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9G DUF603 domain containing protein Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9H Chemotaxix protein CheW Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 9I variable large protein Borrelia specific peptides identified in the urine of Borreliosis patients and selected for MRM analysis. Peptides allow the detection of US and European species: Borrelia: afzelii (i.e., Borrelia afzelii), burgdorferi, bissettii, garinii, spielmanii, valaisiana, hermsii, parkeri, turicatae, hispanica, persica, crocidurae, miyamotoi.

FIG. 10 shows Borrelia proteins identified in the urine of patients have the following functions: biosynthesis, cell cycle regulation, cell wall organization/biogenesis, chemotaxis and motility, DNA replication, recombination, and repair, immune evasion, metabolism, signal transduction, transcription, translation and protein processing, transmembrane lipid and ion transport.

FIG. 11 shows a collapsible cup containing an affinity net for biomarker capture, concentration and preservation (A-F) was validated for immunoassay and mass spectrometry analysis. The cup was developed under NIH funding (R21AI138135 and R21HD097472) and verified to preserve molecular content of urine for immunoassay and mass spectrometry analysis.

FIG. 12 shows a proposed hypotheses on biomarker origin and shedding mechanism.

FIG. 13 classifies pathogen. (A) Frequency of pathogen identification in late stage TBI patients. (B) Borrelia was the most represented genus, followed by Babesia, Bartonella, Ehrlichia and Anaplasma. (C) Frequency of identified proteins.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have meanings that are commonly understood by those of ordinary skill in art. Further, unless otherwise required by context, singular terms shall include pluralities, and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded with the widest scope encompassing numerous alternatives, modifications and equivalents consistent with the principles and features disclosed. For the purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

“Biofluid” or bodily fluids, or body liquids or similar term is a liquid found within a biological system of a living organism. It includes various fluids like blood, lymph, cerebrospinal fluid, synovial fluid, urine, and saliva. These fluids are involved in various processes such as nutrient transport, waste removal, lubrication, and immune responses. In medical and scientific contexts, studying biofluids helps in understanding health conditions and diagnosing diseases.

For example: urine is an attractive biofluid because of its non-invasive nature and because it integrates biomarker abundance over time, given the physiology of urine formation [Magni, Liotta]. In fact, the human blood volume is filtered through the kidney every five minutes under resting conditions [Anatomy & Physiology (openstax.org/details/books/anatomy-and-physiology)]. Thus, circulating biomarkers, which may be intermittently released in blood, are filtered in the kidneys, concentrated and integrated in the bladder, and secreted in the urine.

In an embodiment, bodily fluids can be fluids isolated from anywhere in the body of the subject, such as, for example, a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, intra-organ system fluid, ascitic fluid, tumor cyst fluid, amniotic fluid, cell culture supernatant, and combinations thereof. Biological samples can also include fecal or cecal samples, or supernatants isolated therefrom.

In an embodiment, body fluid could be of subjects such as human or non-human subjects, such as, for example, a rodent, a non-human primate, a companion animal (e.g., cat, dog, horse), and/or a farm animal (e.g., chicken).

The term “subject” is intended to include all animals shown to, or expected to, have nucleic acid-containing particles. In an embodiment, the subject is not a mammal, such as avians, reptiles, etc. In an embodiment, the subject is a mammal, a human or nonhuman primate, a dog, a cat, a horse, a cow, other farm animals, or a rodent (e.g. mice, rats, guinea pigs, etc.). A human subject may be a normal human being without observable abnormalities, e.g., a disease. A human subject may be a human being with observable abnormalities, e.g., a disease. The observable abnormalities may be observed by the human being himself, or by a medical professional. The term “subject,” “patient,” and “individual” are used interchangeably herein.

‘Disease’ as referred herein refers to an abnormal condition that adversely affects the structure or function of all or part of an organism and is not immediately due to any external injury. More particularly, it refers to a condition that impairs the normal functioning of the body such as causing pain, dysfunction, distress, social problems, or death to the person affected, or causing similar problems for those in contact with the impaired subject. A disease may be caused by external factors, such as pathogens, or by internal dysfunctions. For example, pathogens include viruses, bacteria, fungi, protozoa, multicellular organisms, and aberrant proteins known as prions. Internal dysfunctions of the immune system can, for example, produce a variety of different diseases, including various forms of immunodeficiency, hypersensitivity, allergies, and autoimmune disorders. A type or sub-type of a disease is not limited herein, and one or more embodiments could be used to diagnose any type or sub-type of a disease as contemplated by a person skilled in the art. Examples of disease include, without limitation, cancer, etc. In one aspect, the condition or disease is a Borrelia infection or Lyme disease. In one embodiment, the Borrelia infection or Lyme disease is caused by Borrelia burgdorferi, Borrelia afzelli, or Borrelia garinii.

“Biomarker” is synonymously used with biological marker which is a measurable characteristic of a biological state or condition. It is a characteristic that can be objectively measured and evaluated as an indicator of a biological state or condition such as biological processes, pathogenic processes, and/or responses to therapeutic interventions. Biomarkers in a biological samples and/or body fluids, including organs, blood, urine, saliva, tissues, peritoneal fluid, cerebrospinal fluid, cell/bacterial culture supernatant, cervical swab, buccal swab, breast milk and other bodily fluids. Examples of biomarkers, without limitation, include nucleic acids, peptides, proteins, lipids, antigens, carbohydrates, lipid or glycanproteins, exososomes, extracelluar vesicles. Nucleic acid comprises DNA and/or RNA. The nucleic acids can be single stranded or double stranded. Examples of RNA include messenger RNAs, long non-coding RNAs, transfer RNAs, ribosomal RNAs, small RNAs (non-protein-coding RNAs, non-messenger RNAs), microRNAs, piRNAs, snRNAs, snoRNAs, and Y-RNAs. RNA comprise mRNA, miRNA, snoRNA, snRNA, rRNA, tRNA, siRNA, hnRNA or shRNA. In an embodiment, a target biomolecule is a biomarker.

In an embodiment, biomarkers are proteins. In some embodiments proteins are emphasized because they can provide unique insights into diagnosis due to their functional role in mediating disease phenotype [doi.org/10.1080/14789450.2021.1950536].

In an embodiment, biomarkers are unambiguously attributable to Borrelia strains.

In an embodiment, this invention is a breakthrough since it introduces a novel concept in separation/detection technology using a filamentous network for molecular capturing of biomarkers in biological samples.

“Filamentous material” or similar term refers to a thread like material or filament. Filamentous material could be a sole filament or multi-filaments. The filamentous material is selected from natural or synthetic fibers, including but not limited to cellulose, silk, or polymer-based materials. Filaments could be woven, non-woven, interlaced, perforated, penetrable, braided, net, and a variety of other structures. Filamentous material could be either natural, such as cotton or silk, or a polymeric material, such as polylactide, polyglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides, polyolefins, PEEK, PTFE, Dacron and their copolymers, modified derivatives of caprolactone polymers, polytrimethylene carbonate, polyacrylates, polyethylene glycol, hydrogels, photo-curable hydrogels, terminal diols, and combinations thereof. In some embodiments, filaments could be treated with chemicals to affect their physical properties and/or their chemical composition.

Different dimensions of filamentous material could be contemplated by a person skilled in the art to perform the disclosed invention in one or more embodiments. In an embodiment, the filamentous material has a diameter of about 1 mm, and length about 2000 ft. In some embodiments, the filamentous material has a diameter of about 0.5 mm, 1 mm, 1.5 mm, 2 mm, 2.5 mm, etc. In some embodiments, the filamentous material has length about 500 ft, 1000 ft, 1500 ft, 2000 ft, 2500 ft, 3000 ft, 3500 ft, 4000 ft, 4500 ft, 5000 ft or more.

In some embodiments, the filamentous material is a biocompatible material. Biocompatibility as recognized in art refers to the property of the material wherein the material is nontoxic to a biological sample. In an embodiment, filaments could be treated with chemicals before the addition of a biological sample.

In an embodiment, the filamentous material has an imbibing property. The imbibing property is linked with the imbibing capacity of a material to absorb liquid (example: water). In an embodiment, a filamentous material has an imbibing capacity of less than 25%, 20%, 15%, 10%, 5%, 2% or 1% or less by its weight. In an embodiment, the filamentous material is substantially incapable of imbibing fluids.

In an embodiment, the filamentous material is a non-imbibing material. Non-imbibing as used herein refers to a property of the material when it is substantially free of absorbing liquid. ‘Substantially free’ as used herein refers to ability to imbibe the liquid less than 0.5% of its weight. In an embodiment, the filamentous material has no imbibing capacity to absorb liquid.

In an embodiment, molecular capturing is mediated and achieved through affinity ligands located on the surface of the filamentous network establishing a tight interaction with the biomarkers present in the biofluid.

The ‘molecule capturing agent’, ‘affinity agent’, ‘affinity capture reagent’, ‘capturing agent’, ‘capture agent’, ‘affinity ligand’, ‘spacer’ or similar terms refer to probes or molecules that bind to classes of biomarkers (such as proteins, protein post-translational modifications (e.g., phosphorylation or glycosylation), lipids, and nucleic acids).

The interaction between the affinity agent and the biomolecule of interest could be covalent or non-covalent interaction. In some cases, the interaction depends on the biomolecule of interest and the affinity agent, for example, antigen and antibody, enzyme and substrate, receptor and ligand, protein and nucleic acid.

In some cases, interaction between the probes and bioanalytes (biomarkers) is in a non-specific way. The defining characteristic of non-specific affinity probes is that they can be used to identify proteins and bioanalytes that are unexpected or unknown beforehand.

Examples of affinity capture molecules include dyes, metal ions, drugs, antibodies, recombinant antibodies, co-enzymes, vitamins, proteins, peptides, aptamers, receptor ligands, lectins, etc.

In an embodiment, dye includes pigment dye. Pigment dyes include colored lake compositions and non-ionic organic pigments include azoic types such as, but not limited to, Pigment Yellow 1, Pigment Yellow 3, Pigment Yellow 12, Pigment Yellow 13, Pigment Yellow 14, Pigment Yellow 17, Pigment Yellow 74, Pigment Yellow 83, Pigment Yellow 97, Pigment Yellow 10, Pigment Orange 5, Pigment Orange 13, Pigment Orange 16, Pigment Orange 19, Pigment Orange 34, Pigment Orange 36, Pigment Orange 43, Pigment Orange 51, Pigment Red 2, Pigment Red 3, Pigment Red 8, Pigment Red 12, Pigment Red 23, Pigment Red 48, Pigment Red 57, Pigment Red 60, Pigment Red 112, Pigment Red 170, Pigment Red 254, Pigment Violet 3, Pigment Violet 19, Pigment Violet 23, Pigment Violet 29, Pigment Violet 32, Pigment Violet 37, Pigment Violet 42, Pigment Violet 50, Pigment Violet 55, Pigment Violet 60, Pigment Blue 15, Pigment Blue 15:1, Pigment Blue 15:2, Pigment Blue 15:3, Pigment Blue 22, Pigment Blue 27,

Pigment Blue 28, Pigment Blue 60, Pigment Blue 61, Pigment Blue 74, Pigment Green 7, Pigment Green 36, Pigment Green 50, Pigment Green 56, Pigment Green 7:1, Pigment Green 8, Pigment Green 10, Pigment Green 18, Pigment Green 36:2, Pigment Green 50:2, Pigment Brown 6, Pigment Brown 23, Pigment Brown 24, Pigment Brown 25, Pigment Brown 29, Pigment Brown 41, Pigment Brown 43, Pigment Brown 57, Pigment Brown 58, Pigment Brown 60.

In an embodiment, dyes include direct, sulfur, reactive, insoluble azoic, phthalocyanine, acid, metal-complex, basic, and disperse dyes.

In an embodiment, direct dyes comprise sulfonated azo compounds with the general formula R1—N═N—X—N═N—R2 where R1 and R2 are —NH₂SO₃H, —NH₂, —OH, —COOH, —N+(CH₃)₃ substituted aromatic or heterocyclic ring, and N═N, X is the chromophore moiety including, but not limited to, azo, stilbene, oxazine, thiazole, quinonoid, or phthalocyanine groups.

Examples of direct dyes include, but not limited to: Congo red, Direct Brown, Diazine Black BG, Chrysiohenine, Diazine Fast Yellow 4GL, Diazine Fast Blue, Direct Green 6, Direct Brown B, Direct Black 38, Direct Blue 1, Direct Orange 26, Direct Red 81, Direct Blue 86, Direct Yellow 4, Direct Orange 26, Direct Orange 34, Direct Orange 39, Direct Orange 102, Direct Orange 108, Direct Red 16, Direct Red 31, Direct Red 80, Direct Red 81, Direct Red 83, Direct Red 89, Direct Red 227, Direct Blue 1, Direct Blue 15, Direct Blue 71, Direct Blue 86, Direct Blue 199, Direct Green 1, Direct Green 6, Direct Green 26, Direct Black 19, Direct Black 22, Direct Black 38, Direct Brown 2.

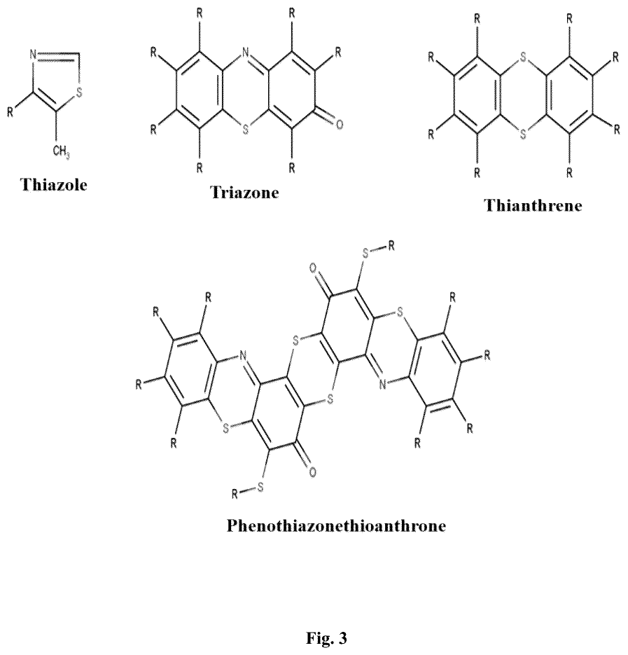

In an embodiment, sulfur dyes comprise dyes with sulfur in the form of (—S—), disulfide (—S—S—), and polysulphides (—Sn—) linking heterocyclic rings. Heterocyclic rings comprise thiazoles, thiazones, thianthrenes, and phenothiazonethioanthrone as shown in FIG. 3. In FIG. 3, R groups include —H, -alkyl group of one or more carbons, examples of alkyl groups comprise, but not limited to, methyl (CH₃—), ethyl (C₂H₅—), propyl (C₃H₇—), butyl (C₄H₉—), —OH, —NH₂, —NO₂, and —Cl.

Examples of direct dyes include, but are not limited to, Methylene Blue, Azure A, Azure B, Methylene Green, Sulfur Black 1, Sulfur Green, Sulfur Brown, Sulfur Yellow G, Vat Yellow 2.

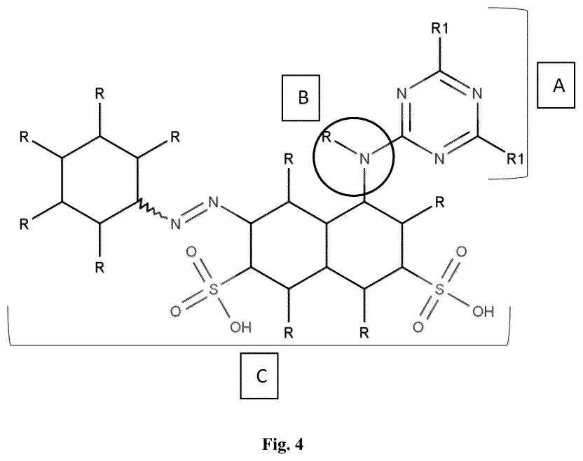

In an embodiment, reactive dyes comprise dyes with the structure C—B-A as shown in FIG. 4, wherein C represents one or more heterocycles linked by a diazo group (—N═N—) where —R represents —H, -alkyl group of one of more carbons, examples of alkyl groups comprise, but not limited to, methyl (—CH₃), ethyl (C₂H₅—), propyl (C₃H₇—), butyl (C₄H₉—), —OH, —NH₂, —NO₂, —SO₃Na, or —Cl. B, a bridge linking the heterocycles from C and the A group of mono, dichlorotriazide group comprising the structure-NH—. A represents a mono or dichlorotriazide ring where R1 represents —Cl or —H, -alkyl group of one of more carbons, examples of alkyl groups comprise, but not limited to, methyl (CH₃—), ethyl (C₂H₅—), propyl (C₃H₇—), butyl (C₄H₉—), or —OH, —NH₂, substituted heterocycle, or another C group supported through a stilbene group. Second C group connected by the stilbene group poses all/some the characteristics described above conforming symmetric bifunctional of asymmetric bifunctional dyes.

Examples of reactive dyes include but not limited to Reactive Orange 1, Reactive Red 3, Reactive Violet 2, Reactive Blue 4, Reactive Red 6, Reactive Blue 5, Reactive Yellow 17, Reactive Yellow 4, Reactive Yellow 2, Reactive Red 2, Reactive Yellow 1, Reactive Orange 4, Reactive Yellow 5, Reactive Orange 13, Reactive Yellow 5, Reactive Orange 13, Reactive Orange 16, Reactive Violet 4, Reactive Black 5, Reactive Blue 4, Reactive Red 120, Reactive Blue 21, Reactive Blue 221, Reactive Red 66, Reactive Blue 19, Reactive Yellow 84, Reactive Yellow 81/105, Reactive Yellow 105, Reactive Yellow 135, Reactive Yellow 84, Reactive Red 120, Reactive Red 141, Reactive Red 152, Reactive Orange 84, Reactive Orange 94, Reactive Green 19, Reactive Blue 71, Reactive Blue 171, Reactive Blue 172, Reactive Blue 160, and Reactive Blue 198.

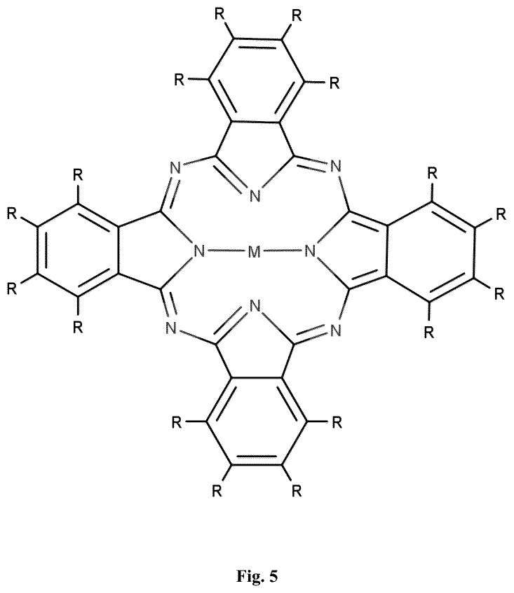

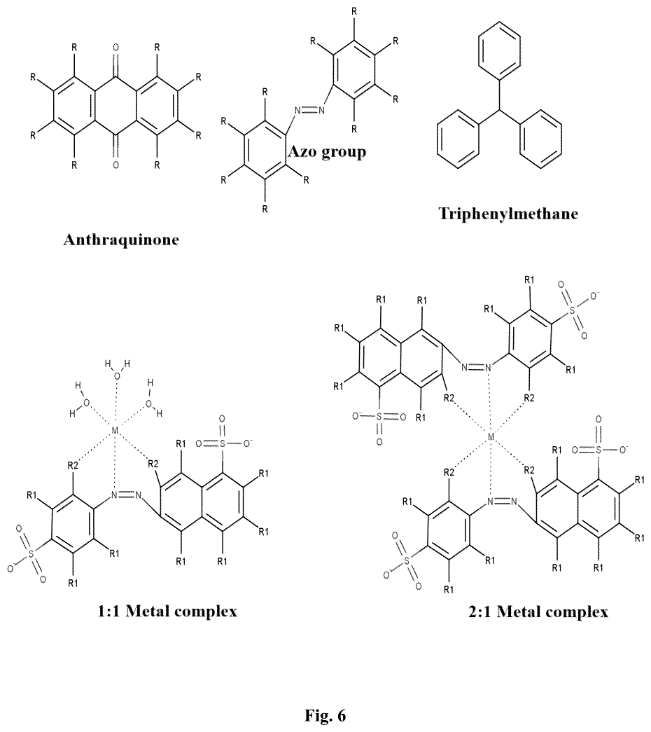

FIG. 5 shows phthalocyanine dyes comprising tetraazo or a derivative of tetrabenzoporphyrin consisting of four isoindole units connected by four nitrogen atoms. 16-membered ring with carbon and nitrogen intercalating can be coordinating metals including, but not limited to, Cu (copper), Zn (zinc), Mg (magnesium), Al (aluminium), Fe (iron), Ni (niquel), Co (cobalt), Pb (lead), Mn (manganese), Ti (titanium), V (vanadium), Cr (Chromium), Pd (Palladium), Pt (Platinum), where —R is —H, -alkyl group of one of more carbons, examples of alkyl groups comprise, but not limited to, methyl (CH₃—), ethyl (C₂H₅—), propyl (C₃H₇—), butyl (C₄H₉—), —NO₂, —SO₃Na, —NH₂, aryl including, but not limited to, phenyl, naphthyl, biphenyl, alkoxy including, but not limited to, methoxy, ethoxy, propoxy, carboxyl group, hydroxy or halogen. Examples of phthalocyanine dyes include, but not limited to, Alcian Blue pyridine variant, Copper Phthalocyanine (CuPc), Nickel Phthalocyanine (NiPc), Cobalt Phthalocyanine (CoPc), Iron Phthalocyanine (FePc), Zinc Phthalocyanine (ZnPc), Manganese Phthalocyanine (MnPc), Titanium Phthalocyanine (TiPc), Vanadyl Phthalocyanine (VO (Pc)), Chromium Phthalocyanine (CrPc), Palladium Phthalocyanine (PdPc), Platinum Phthalocyanine (PtPc), Copper Hexadecafluorophthalocyanine (F₁₆CuPc), Nickel Hexadecafluorophthalocyanine (F₁₆NiPc), Cobalt Hexadecafluorophthalocyanine (F₁₆CoPc), Iron Hexadecafluorophthalocyanine (F₁₆FePc), Zinc Hexadecafluorophthalocyanine (F₁₆ZnPc), Copper Tetrafluorophthalocyanine (F₄CuPc), Nickel Tetrafluorophthalocyanine (F₄NiPc), Cobalt Tetrafluorophthalocyanine (F₄CoPc), Iron Tetrafluorophthalocyanine (F₄FePc), Zinc Tetrafluorophthalocyanine (F₄ZnPc), Copper Octafluorophthalocyanine (F₈CuPc), Nickel Octafluorophthalocyanine (F₈NiPc), Cobalt Octafluorophthalocyanine (F₈CoPc), Iron Octafluorophthalocyanine (F₈FePc), Zinc Octafluorophthalocyanine (F₈ZnPc), Copper Octabromophthalocyanine (Br₈CuPc), Nickel Octabromophthalocyanine (Br₈NiPc), Cobalt Octabromophthalocyanine (Br₈CoPc).

In an embodiment, acid dyes comprise azobenzene (Ph-N═N-Ph), anthraquinone, premetallized (1:1 metal complex and 2:1 metal complex) and triphenylmethane structures. Exemplary is shown in FIG. 6.

Azobenzene (Ph-N═N-Ph) consists of two phenyl groups where —R represents —H, -alkyl group of one of more carbons, examples of alkyl groups comprise but not limited to methyl (CH₃—), ethyl (C₂H₅—), propyl (C₃H₇—), butyl (C₄H₉—), —OH, —NH₂, —NO₂, —SO₃Na.

Anthraquinone is a planar structure consisting of two aromatic rings joined by two carbonyl groups (keto group) forming a central ring. In the structure-R represents —H, -alkyl group of one of more carbons, examples of alkyl groups comprise but not limited to methyl (CH₃—), ethyl (C₂H₅—), propyl (C₃H₇—), butyl (C₄H₉—), —OH, —NH₂, —NO₂, —SO₃Na.

In an embodiment, premetallized dyes comprise a monoazo structure with 1:1 metal complex or 2:1 metal complex where a 1:1 complex contains one metal coordinating one monoazo structure, and 2:1 complex contains one metal coordinating two monoazo structures. In the structures-R1 represents —H, -alkyl group of one of more carbons, examples of alkyl groups comprise but not limited to methyl (CH₃—), ethyl (C₂H₅—), propyl (C₃H₇—), butyl (C₄H₉—), —OH, —NH₂, —NO₂, —SO₃Na, and —R2 represents hydroxyl, carboxyl or amino group, and M represents the metal and chelating site where coordinating occurs. Examples of metals include Cr (chromium), Co (cobalt), Cu (copper), Ni (nickel).

In an embodiment, triphenylmethane structure comprises a triaryl methane skeleton where —R represents —H, -alkyl group of one of more carbons, examples of alkyl groups comprise but not limited to methyl (CH₃—), ethyl (C₂H₅—), propyl (C₃H₇—), butyl (C₄H₉—), —OH, —NH₂, —NO₂, —SO₃Na, halogen, hydroxyl, carboxyl, or amino group (primary, secondary, tertiary and quaternary).

Examples of acid dyes include but not limited to Acid Orange 7, Acid Brown 14, Acid Red 87, Acid Red 27, Acid Blue 119, Fuchsin Acid, Acid Blue 113, Acid Green 50, Acid Green 25, Acid Red 88, Acid Red 151, Acid Blue 1, Acid Orange 10, Acid Red 92, Acid Green 1, Acid Blue 129, Acid Black 48, Acid Black 1, Acid Blue 83, Acid Violet 17, Acid Blue 90, Acid Red 13, Eosin Y, Acid Blue 62, Eosin, Azofuchsin, Alizarin rubinol R, Acid Black 1, Acid Blue 22, (www.chemicalbook.com/ProductCatalog_EN/161119-2.htm).

In an embodiment, basic dyes comprise free or substituted-NH₂ groups in their structure, including but not limited to-NH₂, —N(CH₃)₂, and —N(C₂H₅)₂. Within the basic dyes group, the following classes can be distinguished: i) azo, ii) diphenylmethane, iii) triphenylmethane, iv) acridine, v) xanthene, vi) azine, vii) oxazine, and viii) thiazine.

Azo basic dyes include dyes that contain an azo group (N═N) as a central figure responsible for the chromophoric nature of the dye, aromatic rings with substituents such as amino (—NH₂), hydroxyl (—OH), methyl (—CH₃), and ethyl (—CH₂CH₃) groups. Examples of azo basic dyes are but not limited to aniline yellow, Food yellow 3 and Food yellow 4.

Basic diphenylmethane dye structure is characterized by fused aromatic rings (phenyl rings) through a methylene (—CH₂) bridge. Attached to one or both phenyl rings are positively charged amino groups (—NH₂) and/or methyl (—CH₃), and/or ethyl (—CH₂CH₃) groups. Examples of diphenylmethane dyes are but not limited to Basic Yellow 2, Basic Yellow 3

Basic triphenylmethane dye structure is characterized by the incorporation of three aromatic rings connected through a carbon bridge. Attached to one and/or two and/or three phenyl rings are positively charged amino groups (—NH₂). Auxiliary moieties such as hydroxyl (—OH) and/or methyl (—CH₃), and/or ethyl (—CH₂CH₃) groups may be also incorporated on one or more phenyl rings. Examples of triphenylmethane dyes are but are not limited to Basic Green 4, Pararosaniline, and Homorosaniline.

Basic acridine dye structure is characterized by an acridine nucleus comprising two fused aromatic rings: a phenyl ring (C₆H₄) and a pyridine ring (C₇H₅N). Moieties such as amino groups (—NH₂), positioned in phenyl and/or the pyridine ring, contribute to the positive charge of the molecule. Other moieties include hydroxyl (—OH), methyl (—CH₃), ethyl (—CH₂CH₃), and substituted amines. Examples of acridine dyes are but are not limited to Basic Orange R,

Basic xanthene dye structure is characterized by a xanthene nucleus comprising two benzene rings (C₆H₄) adjoining a central oxygen atom. Moieties such as amino groups (—NH₂), positioned in phenyl and/or the pyridine ring, contribute to the positive charge of the molecule. Other moieties include hydroxyl (—OH), methyl (—CH₃), ethyl (—CH₂CH₃), substituted amines, carboxylic acid (—COOH), and phenyl group (—C₆H₄). Examples of xanthene dyes are but are not limited to Rhodamine B, Rhodamine 6G.

Basic azine dye structure is characterized by an azine nucleus comprising two nitrogen atoms connected through a double bond. Moieties such as amino groups (—NH₂), positioned in phenyl and/or the pyridine ring, contribute to the positive charge of the molecule. Other moieties include hydroxyl (—OH), methyl (—CH₃), ethyl (—CH₂CH₃), substituted amines, carboxylic acid (—COOH), and phenyl group (—C₆H₄). Examples of azine dyes are, but are not limited to, Safranin T.

Basic oxazine dye structure is characterized by an oxazine nucleus comprising a six-membered wing containing two nitrogen atoms and one oxygen. Moieties such as amino groups (—NH₂), positioned in phenyl and/or the pyridine ring, contribute to the positive charge of the molecule. Other moieties include hydroxyl (—OH), methyl (—CH₃), ethyl (—CH₂CH₃), substituted amines, carboxylic acid (—COOH), and phenyl group (—C₆H₄). Examples of azine dyes are, but are not limited to, Basic Blue 6 and Basic Blue 4.

Basic thiazine dye structure is characterized by an oxazine nucleus comprising a six-membered ring containing one nitrogen atom and one sulfur. Moieties such as amino groups (—NH₂), positioned in phenyl and/or the pyridine ring, contribute to the positive charge of the molecule. Other moieties include hydroxyl (—OH), methyl (—CH₃), ethyl (—CH₂CH₃), substituted amines, carboxylic acid (—COOH), and phenyl group (C₆H₄). Examples of azine dyes are, but are not limited to, Basic Blue 9 and Basic Green 5.

In an embodiment, an affinity agent is a hydrogel. Hydrogel is a network of hydrophilic polymer chains that can hold a large amount of water. In an embodiment, hydrogel can absorb at least 10%, 20%, 40%, 60% or more volume of water.

In an embodiment, we use a filamentous material comprised of the non-imbibing solid support (polyamide or nylon 6 fibers) functionalized with synthetic dyes (affinity net) for the capture and concentration of protein-containing urine/plasma fractions to be analyzed with mass spectrometry.

In an embodiment, the filamentous material is functionalized to become a positively charged surface. In some embodiments, the filamentous material is functionalized to become negatively charged.

### Functionalization of Affinity Agents to the Filamentous Material

In an embodiment, affinity agents attach to the filamentous material via a covalent binding (e.g., through one or more carbon-carbon bonds, carbon-nitrogen bonds, carbon-oxygen bonds, etc., either directly or indirectly), or non-covalent binding. Examples of non-covalent binding include affinity, ionic, van der Waals (e.g., dipole/dipole or London forces), hydrogen bonding (e.g., between polynucleotide duplexes), and hydrophobic interactions. In an embodiment, the affinity agents attach to the filamentous material via adsorption process. Any other suitable means for chemical coupling of affinity agents to the filamentous material can be used in the processes described herein.

In an embodiment, the incubation period for the material, e.g., filamentous material, and the affinity agent, e.g., dye, is in a range between about 15 mins to up to about 24 hours, 20 mins to up to 12 hours, or a time period about 15 mins, 30 mins, 60 mins, 1 hr, 2 hrs, 4 hrs, 6 hrs, 8 hrs, 10 hrs or more to functionalize the material with the affinity agent. In some embodiments, the incubation for the material, e.g., filamentous material, and the affinity agent, e.g., dye, is performed at a room temperature (a temperature of from 59° to 77° F. (15° to 25° C.) that is suitable for human occupancy and at which laboratory experiments are usually performed). In some embodiments, the incubation for the material, e.g., filamentous material, and the affinity agent, is performed at about 4° C. In some embodiments, the incubation for the material, e.g., filamentous material, and the affinity agent, e.g., dye, is performed at a temperature of about 10° C., 20° C., 25° C., 30° C., 40° C., 50° C., 60° C., 70° C., 120° C., 140° C. or more. In some embodiments, the incubation for the material, e.g., filamentous material, and the affinity agent, e.g., dye, is performed with or without rotation. For example, without limitation 3-amino-N-methylphthalimide in glycerol showed excitation of the dye molecule. It caused not only shift of the fluorescence spectrum in time but also additional rotation of the dye molecule. This effect, which may be called “wavelength-dependent rotation”, depends on the light frequency of both excitation and fluorescence. (Ref.: Journal of Fluorescence, 2, 81-92 (1992))

In an embodiment, the filamentous material functionalized with the affinity agents may need to be washed to remove unbound affinity agents. This washing step is done using a washing solution. In some embodiments, detergents are added to the washing solution. Detergents suitable for use include, but are not limited to, sodium dodecyl sulfate (SDS), Tween-20, Tween-80, Triton X-100, Nonidet P-40 (NP-40), Brij-35, Brij-58, octyl glucoside, octyl thioglucoside, CHAPS or CHAPSO. In some embodiments, detergents like 0.01-0.5% Triton x-100 are used to remove unbound affinity agents. In some embodiments, reducing agents like 0.1-5% DTT or 0.5-8% 2-Mercaptoethanol are used to remove the unbound affinity agents from the filamentous material.

In an embodiment, the amount of the bound affinity agent to the filamentous material is measured. Measuring instruments, without limitation, include spectrometers. In an embodiment, weight ratio (% W/W) between a total amount of affinity agents attached to the non-imbibing filamentous material is about 0.5 to 2%. In an embodiment, weight ratio of the total amount of affinity agent attached to the non-imbibing filamentous material is about 0.5%, 1%, 1.5%, 2%, 2.5%, 4%, 5%, 7%, 10%, 20% or more.

In some embodiments, the functionalization of the filamentous material changes the imbibing capacity of the material. In some embodiments, the functionalization of the filamentous material decreases the imbibing capacity of the material. In an embodiment, the functionalized filamentous material with affinity agents has an imbibing capacity of less than 25%, 20%, 15%, 10%, 5%, 2%, 1%, 0.5% or less by its weight. In an embodiment, the functionalized filamentous material is substantially incapable of imbibing fluids. In an embodiment, the filamentous material has no imbibing capacity to absorb liquid.

In some embodiments, filamentous network functionalized with capturing agents are collectively referred to as affinity material. In an embodiment, affinity agents functionalized on the filamentous material form an affinity net like structure.

Contacting Biofluids with Functionalized Filamentous Material with Affinity Agents

The affinity net is contacted with the biofluid. In some embodiments, the biofluid is used with or without pre-processing of the sample. Preprocessing steps may include, without limitation, centrifuging, filtering, or pre-clearing the biofluid with a non-functionalized population of beads, and/or depleting any abounded proteins from the biofluid.

In an embodiment, the functionalized filamentous material is contacted with a volume of a biological fluid to allow formation of a complex between the affinity agent and biomarkers present in the biological fluid in a condition suitable to form such complex. Contacting affinity agents with the biomarkers allows affinity agents to sequester the biomarkers present in biofluids.

A person skilled in art would understand that a specific volume of a bodily fluid may not be necessary to perform the invention disclosed in at least one embodiment. For example, if the bodily fluid is too little, then, in some embodiments it is possible to dilute the fluid to accomplish the task disclosed in at least one embodiment.

In an embodiment, conditions suitable for formation of the complex include, for example, pH, temperature, buffer and/or incubation time, etc. For example, if urine is biofluid, then its suitable pH is about 5.5, 6, 6.5, 7 or 7.5. A suitable condition for a biofluid is decided according to the type of biofluid and/or affinity agent, as understood by a person skilled in the art. In an embodiment, pH of the biofluid is around 6.5 to 7.5. In an embodiment, pH is around 5.5 to 6.5.

In an embodiment, an incubation time for biomarkers of the biofluid and the affinity agents is in a range between about 15 mins to up to about 12 hours, 20 mins to up to 12 hours, or a time period about 15 mins, 30 mins, 60 mins, 1 hr, 2 hrs, 4 hrs, 6 hrs, 8 hrs, 10 hrs or so depending on type of biomarkers and/or affinity agents. In some embodiments, there is no incubation time required for reaction to happen between the biomarker and the affinity agent.

In some embodiments, the incubation for biomarkers of the biofluid and the affinity agents is performed at a room temperature. The incubation temperature for biomarkers of the biofluid and the affinity agents is about 4° C. In some embodiments, the incubation for the material, e.g., filamentous material, and the affinity agent, e.g., dye, is performed with or without rotation.

The affinity agent functionalized on the filamentous material captures and concentrates low abundance peptide/proteins and excludes high-abundance resident proteins such as uromodulin and albumin.

### Sample Collection

In an embodiment, an affinity net could be used to build a one step “Origami”.

In an embodiment, a collection vessel could be used to collect the biofluid. The collection vessel could be having the affinity net functionalized with molecular capturing agents. In an embodiment, the collection vessel is collapsible. In another embodiment, the collection vessel is non-collapsible. In an embodiment, the collection vessel could be of a shape, such as funnel, cup, or other convenient shapes, that allows holding of a fluid.

Collection vessels could be made of any suitable material that does not allow leakage of biofluid.

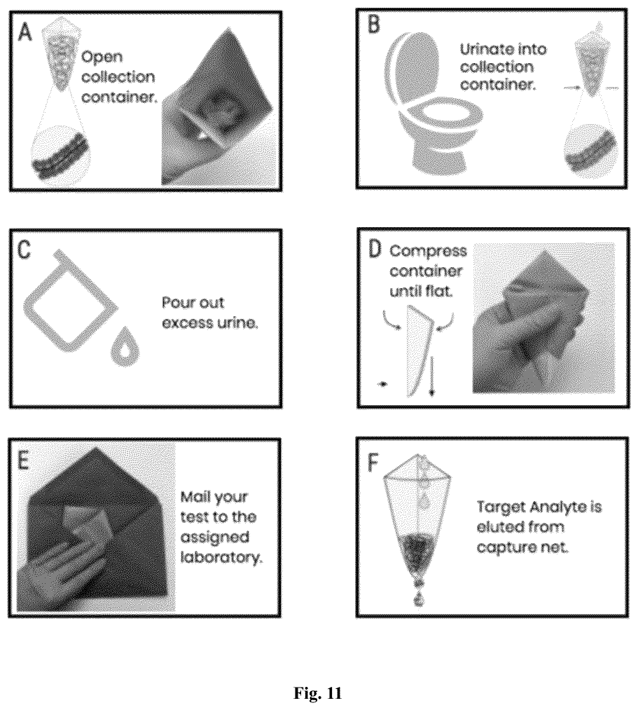

Urine collection cups for home or field collection can compress 60 mL of urinary fluid analytes into a flat envelope for mailing, permitting field or home collection, obviating the need for refrigeration of urine fluid. The origami cup houses the collapsible 3-D affinity capture net that sequesters all of the solution phase analytes in the urine volume. Analytes are then extracted from the net and subjected to mass spectrometry and laboratory analysis (FIG. 11).

In an embodiment, we have built and validated a collapsible urine collection device that incorporates a novel biomaterial (affinity network) engineered to perform rapid molecular recognition, capture, concentration and preservation of target proteins (FIG. 11)¹⁻⁴. The cup achieves 100-fold amplification of sensitivity and a precision of less than 10% Coefficient of Variation, CV, with a sensitivity of about 2.5 picograms/ml.

In an embodiment, collection vessel achieves at least about 100-fold, 200-fold, 300-fold, 500-fold or more, and a precision of less than 10%, 9%, 8%, 7%, 6%, 5%, 4%, 3%, 2%, 1% of the targeted biomarkers with a sensitivity of about 0.5 picograms/mL, 1 picograms/mL, 1.5 picograms/mL, 2 picograms/mL, 2.5 picograms/mL, 5 picograms/mL, 7 picograms/mL or more.

Captured proteins are protected from enzymatic degradation, supporting the use of the disclosed device for urine shipment devoid of the need of cold chain, climate-controlled transport. Thanks to the support of the NIH and the US Fulbright program, user acceptability explored in different regions of the world (Nepal and Guinea Bissau), the disclosed cup design was optimized to meet patient user acceptability criteria. In an embodiment, collection vessel preserves a targeted protein. In an embodiment, more than 50%, 60%, 70%, 80%, 90%, 95%, 99.5% or more targeted proteins are preserved in the collection vessel.

### Extraction of the Captured Biomarker

In an embodiment, sequestered biomarkers (captured biomarkers) from the filamentous material are eluted. In some embodiments, the elution process comprises extracting the complex, formed between the affinity agent and the at least one biomarker from the biological sample.

In an embodiment, the primary determinant of the yield of biomarker capture is the binding affinity (KD) of the affinity ligand to the target class of analytes⁴, so molecular probes capable of binding classes of bioanalytes with high affinity are preferred. Identification of enriched analytes is performed by the downstream analytical device.

The term “dissociation constant” or “KD” defines the specific binding affinity. As used herein, the term “KD” (usually measured in “mol/L”, sometimes abbreviated as “M”) is intended to refer to the dissociation equilibrium constant of the particular interaction between a first protein and a second protein. In the context of the present invention, the term KD is particularly used to describe the binding affinity between an affinity agent and a biomarker.

In some embodiments, an affinity agent of the invention is considered to bind to a biomarker, if it has a dissociation constant KD to biomarker of at least 1 μM or less, or preferably 100 nM or less, more preferably 50 nM or less, even more preferably 10 nM or less. In some embodiments, an affinity agent binds to a biomarker with a binding affinity (KD) of less than 5 nanomolar (nM). In other embodiments, the affinity agents bind with a K_{D} of less than 4 nM, 3 nM, 2.5 nM, 2 nM or 1 nM. Further, in some other embodiments affinity agents of the invention binds biomarkers with KD of about 5 nM to about 1 nM, or about 5 nM to about 2 nM, or about 5 nM to about 3 nM, or about 5 nM to about 4 nM, or about 3 nM to about 1 nM, or about 2 nM to about 1 nM. In some embodiments of the invention, the affinity agents bind with biomarkers with KD of less than 950 picomolar (pM), or less than 900 pM, or less than 800 pM, 700 pM, 600 PM or 500 pM or less. In some embodiments of the invention, the affinity agents bind with biomarkers with KD of less than 500 picomolar pM), or less than 250 pM, or less than 100 pM, 50 pM, 20 PM or less. KD could be assessed using a method described herein or known to one skilled in the art (e.g., a BIAcore assay, ELISA) (Biacore International AB, Uppsala, Sweden).

The extraction process involves a suitable buffer. In some embodiments of the extraction process, the number and pattern of elution steps is varied depending on the antigen of interest and/or the sensitivity/specificity of the downstream application. In some embodiments, the pH of the elution buffer has pH about 6 to pH 9. In some embodiments, the elution buffer is supplemented with a detergent, for example: with 0.1%-5% tween, 0.01-0.5% Triton x-100. In some embodiments elution buffer is supplemented with reducing agents like 0.1-5% DTT or 0.5-8% 2-Mercaptoethanol are used to remove the non-specifically bound biomarkers.

In some embodiments, the elution buffer is used with or without proteinase, phosphatase or RNAse inhibitors, etc. In some embodiments, the vortex is replaced by sonication, freeze-thaw cycle or any physical perturbation.

In some embodiments, the extraction process disclosed herein comprises eluting the complex in an intact form. In some embodiments, the extraction process disclosed herein comprises eluting the biomarker in an intact form. In some embodiments, the extraction step disclosed herein comprises extracting one or more nucleic acids, proteins, carbohydrates or lipids from the complex/captured biomarker.

Captured biomarkers such as proteins are eluted in a smaller volume to achieve an effective 1,000-fold concentration of proteins^{4,5}. In another embodiment, captured proteins could be concentrated to at least 500-fold, 750-fold, 1000-fold, 1500-fold, 2000-fold, 2500-fold or more.

In some embodiments, one or more concentration steps are performed to reduce the volumes of sample, before or after contacting the biofluid with the capture surface. Concentration after contacting the biofluid with the affinity agents may be through centrifugation of the sample at high speeds, e.g. between 10,000 and 100,000 g, to cause sedimentation of the captured biomarkers.

In some embodiments, the elution process is followed by digestion of captured biomarkers. In some embodiments, the digestion and elution process are simultaneous. In some embodiments, digestion may not be required.

The digestion process could involve enzymes (such as ArgC, AspN, chymotrypsin, GluC, LysC, LysN, trypsin, snake venom diesterase, pectinase, papain, alcanase, neutrase, snailase, cellulase, amylase, chitinase or combinations thereof) or chemical reagents (such as hydrochloric acid, formic acid, acetic acid, hydroxide bases, cyanogen bromide, 2-nitro-5-thiocyanobenzoate, hydroxylamine, or appropriate combinations thereof).

In an embodiment, the process involves downstream analysis of the captured biomarkers eluted from the affinity agents. In some embodiments, downstream analysis is performed after digestion of the biomarkers.

In an embodiment, affinity network technology can be incorporated in a novel “origami” collection envelope that folds 100 mL of urine fluid biomarkers into a dry confidential envelop for secure mail service transport, completely obviating the need for liquid or frozen urine handling shipment or storage.

In an embodiment, the affinity net is a polymer wool grafted with a hydrogel containing molecular recognition probes (FIG. 11), housed in a waterproof collapsible collection cup.

### Downstream Analysis

In an embodiment, a downstream measurement assays on proteins, protein modifications, sugars, lipids, RNA, DNA, and metabolites, etc, including, but not limited to, Western Blot, ELISA, qPCR, RNASeq, DNASeq, flow cytometry, immune-fluorescence, immune-gold electron microscopy, and mass spectrometry, and any combination thereof. Any art-recognized techniques for the analysis of the purified populations and/or subpopulations of the extracted biomarkers provided herein are suitable for use in the processes described herein.

“Bottom-up proteomic analysis” is a method to identify proteins and characterize their amino acid sequences and post-translational modifications. Bottom-up proteomics involves proteolytic digestion of proteins into peptides. The peptides are then identified and quantified, and the data is used to infer information about the original proteins.

In an embodiment, a bottom-up proteomic analysis using tandem MS (Mass spectrometer) is accomplished in multiple steps: proteins are extracted from the affinity net using high percentages of denaturants and a degradable detergent and are digested with trypsin before separation by liquid chromatography (LC). Peptides are then eluted from the LC column using gradients of hydrophobic and hydrophilic buffers and introduced into the MS, which generates fragmentation spectra.

In an embodiment, the experimental spectra are then matched with predicted spectra of peptides from in-silico digested proteins using known sequences (such as FASTA database). A scoring algorithm (e.g., Mascot) results in peptide and protein identification and provides a p-value quantifying the probability that the identification happened by chance. Proteomic software (e.g., Proteome Discoverer) provides tools for label-free quantification, such as MS/MS spectral counts or parent MS1 ion chromatographic peak integration. Calculations to match an experimental spectrum to a peptide sequence are very intensive when the size of the protein database increases significantly⁷. Given the many species that were recognized to be pathogenic to humans, the size of the database used in this procedure is large. Peptides shorter than seven amino acids are filtered out to minimize organism attribution by chance, and peptides are assigned to the lowest non-ambiguous taxonomic rank⁷.

In an embodiment, the downstream analysis of a nucleic acid, for example, is to measure and/or compare levels of expression to predetermined thresholds. For example, at least one biomarker (i.e. one or more), such as a group of genes, may be identified as a signature by analyzing clinical samples procured with stringent inclusion and exclusion criteria for the intended clinical utility. On a per-sample basis, a continuous or discrete score may be derived by performing statistical classification analysis including but not limited to random forest, logistic regression and neural network. On this score, a threshold is defined that separates intended sample groups for the clinical utility with an acceptable clinical specificity and sensitivity.

In some embodiments, the methods described herein include one or more in-process controls. In some embodiments, the in-process control is detection and analysis of a reference gene or a reference protein. The reference protein is/are analyzed by additional ELISA or Western blot.

In some cases, native biomarkers are quantified and analyzed.

In an embodiment, analysis of captured biomarkers allows generation of large amounts of marker data from biomarker measurement approaches such as mass spectrometric approaches. In various embodiments, measurements are made so that levels are determined for at least 5, 6, 7, 8, 9, 10, 11, 12, 13, 14, 15, 16, 17, 18, 19, 20, 25, 30, 35, 40, 45, 50, 60, 70, 80, 90, 100, 150, 200, 500, 1000, 2000, 5000, 10,000, 20,000 or more biomarkers in a sample. Various mass spectrometric analysis of samples, such as samples comprising proteins and/or protein fragments, facilitates generation of very large databases from which biomarker levels indicative of a patient health status are derived.

In some embodiments, ‘semi-targeted’ mass spectrometric approach to biomarker measurement is used. Samples are collected as disclosed herein. Prior to mass spectrometric analysis internal standards, for example, heavy-labeled biomolecules, are added to the samples. These biomolecules can co-migrate with, or be adjacent to, particular proteins or polypeptides of interest. As the biomolecules are labeled, they are readily and independently detected in mass spectrometric output. When they are slightly mass-altered relative to the protein or polypeptide which they are targeting for measurement, the biomarkers readily identify the unlabeled target, while migrating at a position that is displaced sufficiently so as to allow the identification of the native protein or polypeptide without obscuring its signal. Such markers are used in some cases to identify proteins or polypeptides of particular interest in a sample, such as proteins recognized by the FDA to circulate in human blood and to be of particular relevance in at least one health status or health condition. Furthermore, heavy-labeled biomolecules provide the means to quantify the absolute abundance of the associated unlabeled target, providing a precise measurement of the target. Thus, approaches herein allow the targeted analysis of particular proteins of interest in a mass spectrometrically analyzed sample. This use of labeled markers to facilitate biomarker quantification and identification in samples allows high throughput, automated biomarker measurement in large numbers of samples as is conducive to database generation.

These approaches do not preclude the concurrent analysis of untargeted mass spectrometric signals in a sample output. That is, the labels identify peaks or signals of interest, but they do not obstruct one from observing or quantifying other unlabeled peaks or signals in a sample. Consequently, in some embodiments one can perform a targeted assay of a set of proteins of interest for which labeled mass-shifted markers are available, while at the same time collect untargeted data relating to up to every detected signal or spot in the mass spectroscopy data output.

In some examples, label, label-free, or any other mass-shifted techniques are used to identify or quantify molecular markers in the sample. For example, label-free techniques include but are not limited to the Stable Isotope Standard (SIS) peptide response. Label techniques include but are not limited to chemical or enzymatic tagging of peptides or proteins. In some examples molecular markers in the sample include all the proteins associated with a particular disease. In some examples, these proteins are selected based on several performance characteristics (i.e., peak abundance, CV's, precision, etc.).

In some embodiments, downstream involves protein reconstruction, homology mapping of all peptide sequences of significance to the Human UniProt DB using a variety of reported methods (Nesvizhskii, A. I.; Keller, A.; Kolker, E.; Aebersold, R. A statistical model for identifying proteins by tandem mass spectrometry. 2003, 75, 4646-4658; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Mujezinovic, N.; Schneider, G.; Wildpaner, M.; Mechtler, K.; Eisenhaber, F. Reducing the haystack to find the needle: improved protein identification after fast elimination of non-interpretable peptide MS/MS spectra and noise reduction. BMC Genomics 2010, 11, S13).

In an embodiment, an algorithm performs peptide authentication, which incorporates stringent filtering criteria in order to minimize the false positive rate. The algorithm includes the following steps: A) Statistical and physical parameters for peptide spectrum matching. B) Unambiguous peptide attributes to one microorganism, thanks to BLAST searches against the NCBI Reference Sequence database (RefSeq), a comprehensive dataset containing the available protein sequence information for any given species. C) Validation of protein database annotation. The full-length protein, to which every peptide is attributed, will be aligned with homologous proteins of evolutionary related organisms in the clade.

In an embodiment, if a full-length protein has greater than 60% identity with proteins in a query, the database annotation is considered valid. In an embodiment, when a full-length protein has greater than 70% identity with proteins in a query, the database annotation is considered valid. In another embodiment, when a full-length protein has greater than 80% identity with proteins in a query, the database annotation is considered valid.

### Application of the Invention:

This invention can be beneficial for patients or scenarios including a) patients with complex or multifactorial diseases, b) patients who present symptoms attributed to multiple conditions, c) patients who were diagnosed and need to be monitored through the diseases progression and treatment response, d) personalized medicine and targeted therapies, biomarker panels are key to identifying patients who may benefit from personalized therapies, e) clinical trials.

The invention will be used for the extraction and analysis of biomarkers in biofluids including fluids from organs, tissue, blood, serum, plasma, urine, tears, interstitial fluid, sweat, peritoneal fluid, saliva, cerebrospinal fluid, cell/bacterial culture supernatant, cervical swab, buccal swab, and/or environmental sample.

The invention will be useful in any diagnostics setting, but particularly useful when refrigeration is not available, or when the sample volume/size is small.

In an embodiment, the present invention is useful in therapeutic effects of drugs, such as vaccines.

In some embodiment, the method described herein provides new insights of biomarkers.

In an embodiment, material such as biocompatible material can potentially improve biomarker discovery and therefore improve/allow:
a) comprehensive assessment: Current tests look at one biomarker at a time, require a high volume of samples and time-consuming tasks. By analyzing a panel of biomarkers, it allows a more comprehensive assessment of multiple disease-related pathways and mechanisms simultaneously.
b) early detection/diagnosis: In some cases, biomarkers can be low, or minimal, at the early stages of the disease, or may exhibit changes that are not easily detectable, predictable, scored, categorized through traditional methods. Overcoming the concentration problem improves the sensitivity of detection by identifying multiple biomarkers over time leading to better treatment outcomes and prognosis.
c) differential diagnosis: Different diseases may share similar symptoms making it challenging to differentiate between them. Tests for each individual disease can be expensive, time-consuming, or even inexistent. Panel-based biomarker analysis can help to rule out medical conditions and identify the specific disease in the same analysis.
d) monitoring disease progression and treatment response: Changes in the biomarker concentration can provide insights about treatment efficacy even when biomarkers are low in concentration.
e) non-invasive and cost-effective procedures: Collecting a biological sample for panel-biomarker discovery is a non-invasive and simple procedure compared to other diagnostic methods. It can eliminate the need for multiple separate tests, leading to cost savings for patients and healthcare systems.

This invention overcomes the following problems/limitations:
a) limited specificity: Biological samples can contain markers that indicate the presence of a disease. However, these markers are often not specific. Capturing multiple markers from a biological sample can increase diagnostic accuracy and enhance accuracy through clinical correlation.
b) sensitivity issues: When the concentration of markers is low, the diagnosis is challenging leading to false negative or inconclusive results.

In an embodiment, the invention increases sensitivity of summative outcomes through biomarker concentration enhancement and biomarker validation. If the biomarkers of interest are in very low concentration, they cannot be detected; isolation and concentration of biomarkers, therefore, enhance detectability. Additionally, using a panel of biomarkers, instead of relying on a single biomarker, improves sensitivity and validation. The panel-based approach enhances diagnostics, increasing sensitivity and specificity. In an embodiment, the present invention has high sensitivity and is able to detect a target peptide present in an amount about 0.5 picograms/mL, 1 picograms/mL, 1.5 picograms/mL, 2 picograms/mL, 2.5 picograms/mL, 5 picograms/mL, 7 picograms/mL or more.

In an embodiment, the present invention has high specificity, such as about 90%, 91%, 92%, 93%, 94%, 95%, 96%, 97%, 98%, 99%, 99%, 99.5%, or more.

In an embodiment, the present invention has high sensitivity, such as about 90%, 91%, 92%, 93%, 94%, 95%, 96%, 97%, 98%, 99%, 99.5%, or more.

In an embodiment, this invention can help differentiate diseases or subtypes, increasing accuracy in the diagnosis. Finally, it can also aid disease monitoring and prognosis once patients are treated by assessing changes in the biomarker panel over time.

In an embodiment, a set of pathogen derived proteins, originating from a diverse set of physiological functions due to ongoing disease, provide information about the active functional state of the pathogen at the time of biomarker shedding.

Preservation of protein signatures can retain proteins in their native state, i.e., preserve their correctly folded, three-dimensional structure, state of association, and functional integrity.

In an embodiment, more than 50% of the total target protein is preserved. In an embodiment, more than 60% of the total target protein is preserved. In an embodiment, more than 70% of the total target protein is preserved. In an embodiment, more than 80% of the total target protein is preserved. In an embodiment, more than 90% of the total target protein is preserved. In an embodiment, more than 95% of the total target protein is preserved.

In an embodiment, analysis includes Multiple Reaction Monitoring (MRM): MRM analysis could be performed using heavy isotope calibrators to achieve a highly linear and precise quantitation^{25,26}.

In an embodiment, analysis includes Custom monoclonal antibody production. In an embodiment, analysis includes Reverse Phase Protein Arrays (RPPA). Selected proteomic biomarkers will be translated to immunoassay for the validation phase. In an embodiment, analysis includes Reverse Phase Protein microarrays (RPMA), first developed in the applicant team's laboratory, are widely used worldwide to conduct quantitative multiplex micro immunoassays on hundreds of microvolume patient samples on a single slide.

In an embodiment, the target proteins captured via molecular capturing technique are preserved. In an embodiment, the target proteins are preserved during the extraction process of the invention. In an embodiment, the target protein is preserved after the extraction process. In an embodiment, the target proteins are preserved during analysis step of the target protein. In an embodiment, the target proteins are preserved during the whole process of the invention. In an embodiment, the target protein is preserved during at least one step of the process.

In an embodiment, the present invention could be used for a direct test for Borreliosis based on pathogen-derived sequence-specific peptides that can identify multiple Borrelia species, and that can distinguish Borreliosis from other febrile illnesses.

This approach is highly sensitive and specific for the following reasons: 1) A novel biomaterial engineered to perform rapid molecular recognition, capture, concentration and preservation of target proteins¹⁻⁴, achieving very high sensitivity of 2.5 picograms/mL; 2) Discovery of a large set of novel pathogen-derived biomarkers, in the urine of well characterized Borreliosis patients using mass spectrometry analysis; 3) Peptide amino acid sequence specificity was insured by a novel bioinformatics pipeline comparing the peptides against all sequenced organisms; 4) A set of pathogen derived proteins originate from a diverse set of physiologic functions ongoing in the Borrelia pathogen, thus providing information about the active functional state of the pathogen at the time of biomarker shedding.

In an embodiment a direct test can identify Borreliosis in early phases preceding seroconversion, and in late stages, in case of pathogen persistence, and can be used to study the consequences of therapy, and its efficacy.

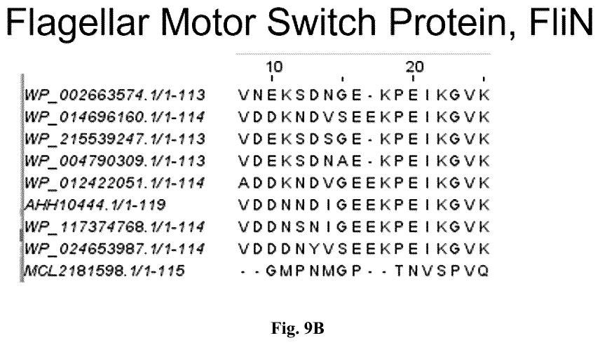

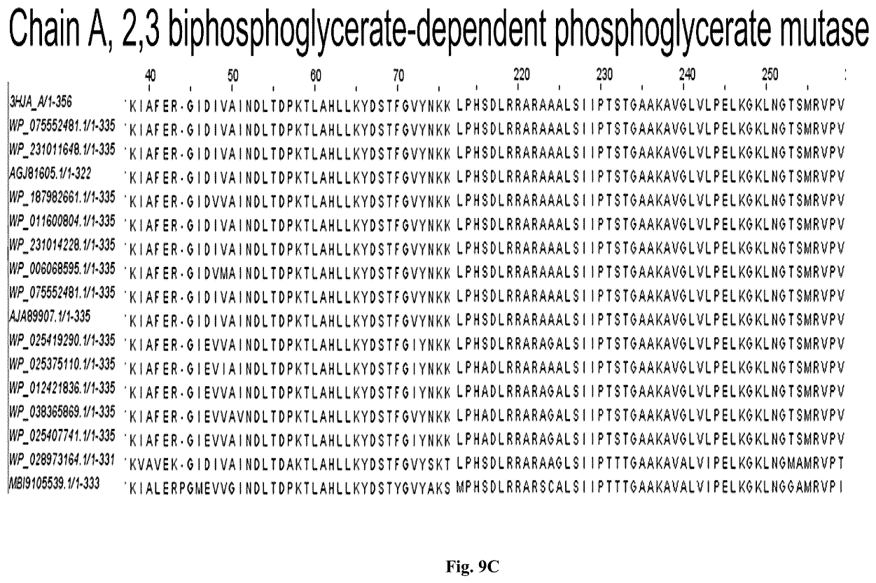

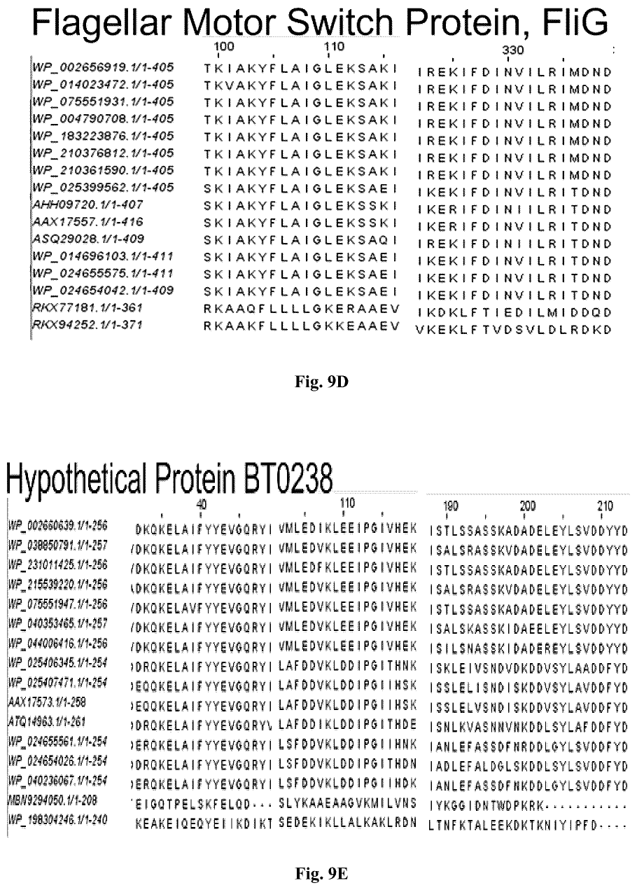

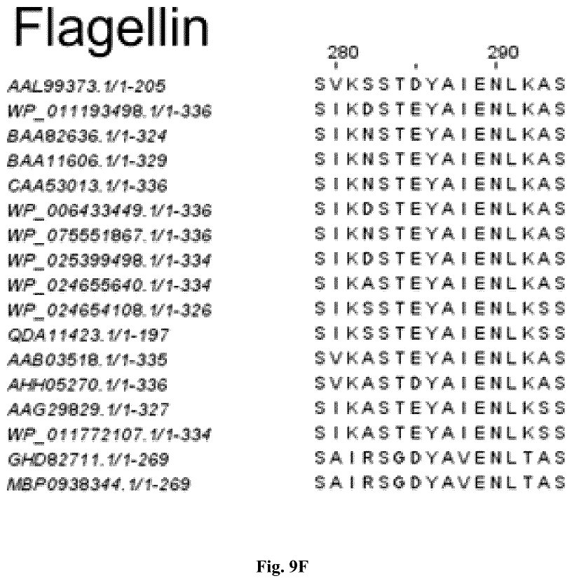

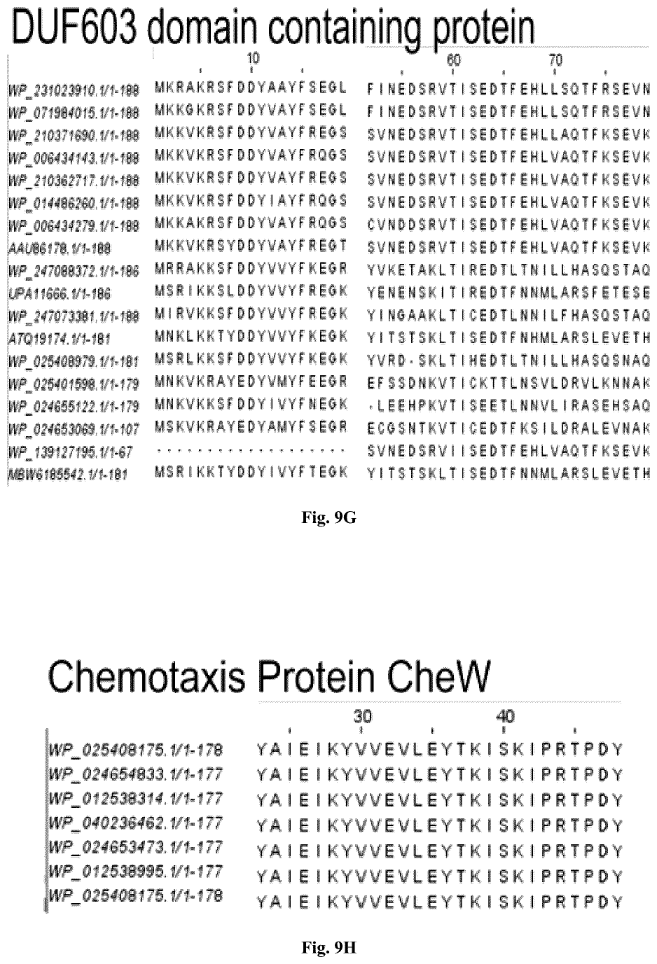

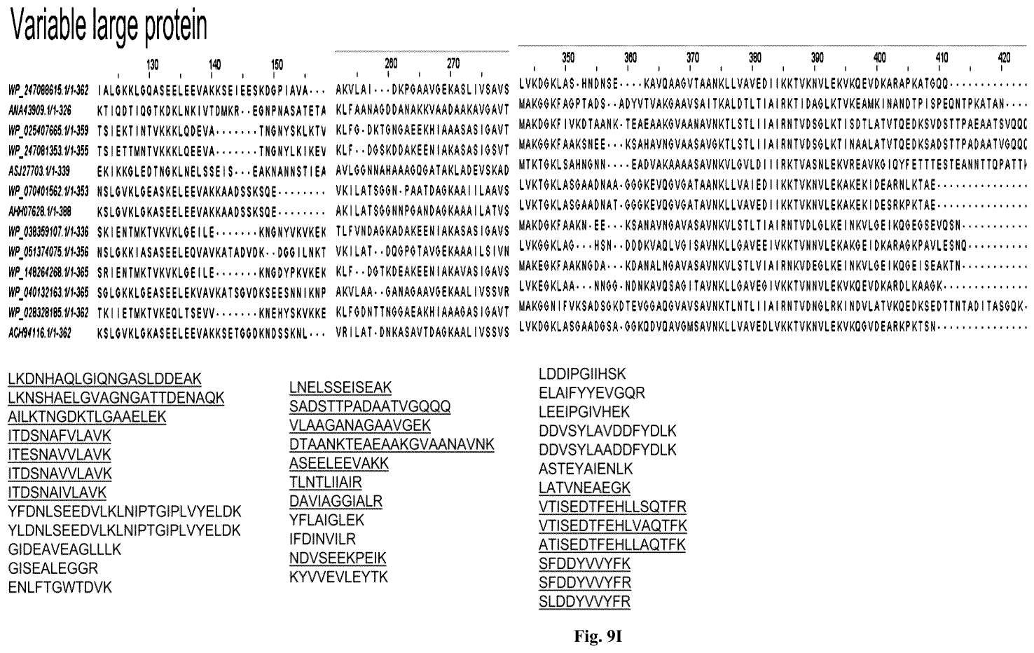

In an embodiment, a panel of 10-20 protein biomarkers that predict Borrelia infection with high sensitivity and specificity (90% and 95%, respectively) will be identified using multiple reaction monitoring (MRM, FIG. 9). This novel MRM panel will be validated in blinded and independent, well characterized, longitudinal clinical samples. Monoclonal antibodies will then be raised against the prioritized biomarker regions authenticated by MRM, and a multiplex immunoassay will be developed.

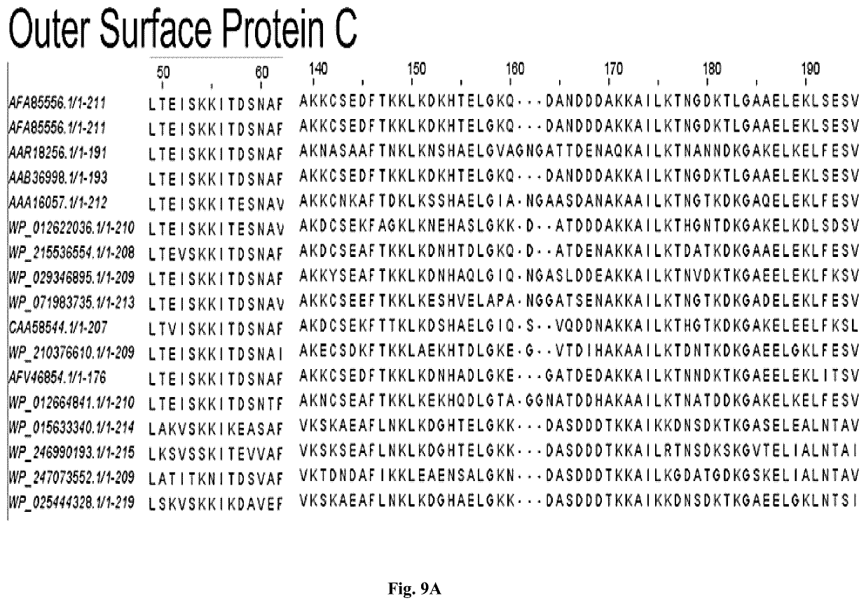

In an embodiment, a long-term envisioned goal is to translate the multiplex assay panel from an LDT test run in the applicant's CAP/CLIA medical diagnostic lab, under an ongoing IRB approved clinical study, to achieve FDA guidance and, potentially, FDA regulatory approval, for the immunoassay for a specific early detection indicated use (FIG. 8). After engaging a commercial partner, and data collection from many independent clinical entities, guidance will be sought from the American Medical Association pursuant to a potential future request for a CPT code.

Kits for Isolating Biomarkers from a Biological Fluid

One aspect of the present invention is further directed to kits for use in the methods disclosed herein. The kit comprises an affinity net sufficient to separate biomarkers from a biological sample from unwanted particles, debris, and small molecules that are also present in the biological sample.

The present invention also optionally includes instructions for using the foregoing reagents in the isolation and optional subsequent nucleic acid and/or protein extraction process. In another aspect, the kit optionally includes instructions for using the foregoing reagents in the isolation and optional subsequent nucleic acid and/or protein extraction process.

The present invention is further illustrated by the following examples that should not be construed as limiting. The contents of all references, patents, and published patent applications cited throughout this application, as well as the Figures, are incorporated herein by reference in their entirety for all purposes.

### Working Example: Identification of Unambiguous Borrelia Peptides in Human Urine Using Affinity Capture and Mass Spectrometry

Borrelia proteins carry out the ongoing pathology functions, including adaptation to therapies, tissue-specific host interactions, immune evasion, and tissue tropism. Treatment decisions concerning acute and persistent Borreliosis are currently based on serology testing and clinical evaluation of the patient's medical history and symptoms⁵. Subjective tools and indirect markers, instead of a definitive molecular diagnosis, contribute to missed or delayed diagnosis, terrible suffering, and long-lasting health consequences for the estimated 476,000 people per year newly affected by the lyme disease. The estimated cost of treating Borreliosis was USD 9.6 billion in 2018, with a significant portion of the cost devolved to late stage disease⁶.

Borreliosis includes low pathogen burden, transient spirochetemia, heterogeneity of Borrelia groups, antigenic variability, tissue tropism, and microenvironment adaptation. The exceedingly low concentration of Borrelia-derived proteins in urine and Borrelia genetic diversity [PMID: 30081938] pose an extraordinary analytical challenge for robust detection [Magni et al, Liotta et al]. Rare proteins in the urine matrix are masked by high abundance resident proteins, such as uromodulin and albumin, and evade identification [doi.org/10.1080/14789450.2021.1950536, Magni et al].

With funding from the National Institutes of Health, National Institute of Allergy and Infectious Diseases, and the Commonwealth of Virginia, we have developed a protein based, direct assay to identify biomarkers, in urine, of Borreliosis and other tick-borne illnesses.

One Borrelia organism, with only one genome, contains many thousands of proteins that carry out the ongoing pathology functions, including adaptation to therapies, tissue-specific host interactions, immune evasion, and tissue tropism. This built-in amplification of specific biomarkers, in addition to the functional insights that the proteins provide, constitutes a strong rationale for proteomic molecules as the biomarker of choice. The assay leverages a combination of innovative nanomaterials, molecular recognition, mass spectrometry analysis and bioinformatics approaches to overcome the issues of low analyte abundance and pathogen biological heterogeneity.

A workflow that combines affinity enrichment, liquid chromatography tandem mass spectrometry (LC-MS/MS) and a bioinformatic pipeline to identify Borrelia peptides in urine is described (FIG. 1). The workflow addresses the issues of low analyte abundance and pathogen biological heterogeneity. Affinity enrichment has been shown to increase detection sensitivity and to reduce analytical variability of proteomic mass spectrometry analysis [doi: 10.1016/j.jprot.2008.06.011, PMID: 29542338].

Affinity enrichment has been shown to increase detection sensitivity and to reduce analytical variability of proteomic mass spectrometry analysis [doi: 10.1016/j.jprot.2008.06.011, PMID: 29542338]. Effective affinity capture reagents are probes that bind classes of bioanalytes such as proteins, protein post-translational modifications (e.g., phosphorylation or glycosylation), lipids, and nucleic acids, in a non-specific way. The defining characteristic of non-specific affinity probes is that they can be used to identify proteins and bioanalytes that are unknown ahead of time. The identification of enriched analytes is performed by the downstream analytical device.

The primary determinant of the yield of biomarker capture is the binding affinity (KD) of the affinity ligand to the target class of analytes [PMID: 29542338], so molecular probes capable of binding classes of bioanalytes with high affinity are preferred. Examples of affinity capture molecules that have been used successfully for mass spectrometry proteomics analysis include dyes, metal ions, and drugs [PMID: 29542338].

In the procedure, we use an affinity material comprised of a non-imbibing solid support (polyamide or nylon 6 fibers) functionalized with synthetic dyes (affinity net) for the capture and concentration of protein containing urine fractions to be analyzed with mass spectrometry. The affinity net, once contacted with large volumes of urine, captures and concentrates low abundance Borrelia proteins and excludes high abundance resident human proteins such as uromodulin and albumin. Captured proteins are then eluted in a smaller volume to achieve an effective 1,000-fold concentration of Borrelia proteins [Magni et al.].

A bottom-up proteomic analysis using tandem MS is accomplished in multiple steps: proteins are extracted from the affinity net using high percentages of denaturants and a degradable detergent, and are digested with trypsin before separation by liquid chromatography (LC). Peptides are then eluted from the LC column using gradients of hydrophobic and hydrophilic buffers and introduced into the MS, which generates fragmentation spectra. The experimental spectra are then matched with predicted spectra of peptides from in-silico digested proteins using known sequences (FASTA database). A scoring algorithm (e.g., Mascot) results in peptide and protein identification and provides a p-value quantifying the probability that the identification happened by chance. Proteomic software (e.g., Proteome Discoverer) provides tools for label-free quantification, such as MS/MS spectral counts or parent MS1 ion chromatographic peak integration [doi.org/10.1016/B978-0-444-63688-1.00012-4].

Calculations to match an experimental spectrum to a peptide sequence are very sophisticated when the size of the protein database increases significantly [PMID: 34917758]. Given Borrelia genetic diversity, and the many species that were recognized to be pathogenic to humans, the size of the database used in this procedure is large, as it includes 19 organisms. An important step after microbial database matching is identifying peptides that are unique to the Borrelia pathogenic species taxonomic group [doi.org/10.3390/biology10101036, doi.org/10.3390/biology10111117] compared to all known organisms. Peptides shorter than seven amino acids are filtered out to minimize organism attribution by chance, and peptides are assigned to the lowest non ambiguous taxonomic rank [PMID: 34917758].

In an embodiment, highly accurate and sensitive measurement of urinary Borrelia derived protein markers in the urine can diagnose active Borrelia infection with high sensitivity and specificity. The concept was validated in a published clinical study, where we applied an unbiased proteomics approach enhanced by affinity capture and bioinformatics analysis to identify tick borne pathogen peptides in the urine of patients under clinical evaluation for tick borne illnesses at different stages. Targeted pathogens were Borrelia, Babesia, Anaplasma, Rickettsia, Ehrlichia, Bartonella, Francisella, Powassan virus, tick-borne encephalitis virus, and Colorado tick fever virus. Specificity was defined by 100% amino acid sequence identity with tick-borne pathogen proteins, evolutionary taxonomic verification for related pathogens, and no overlap with human or other organisms.

In an embodiment, we identified a panel of 160 urinary Borrelia derived sequence-specific protein biomarkers that were detected ex vivo from patient urine that constitute the basis for candidate biomarker validation and test development.

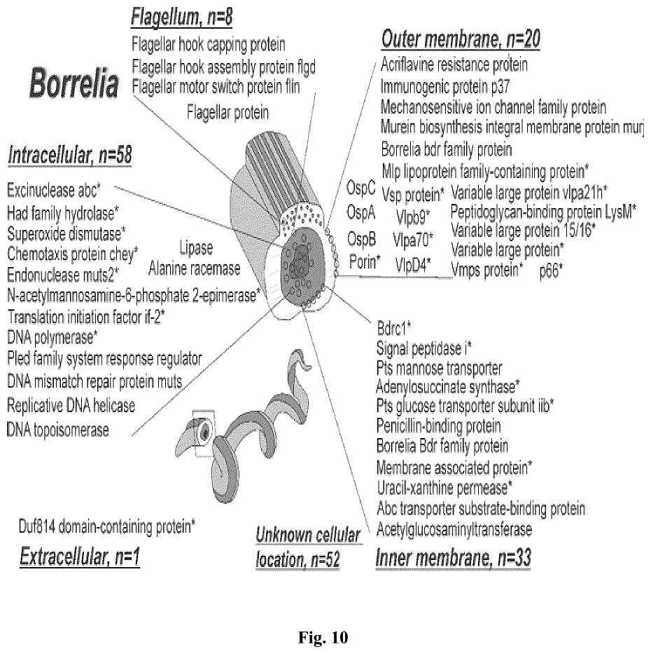

In an embodiment, we identified 160 proteins from Borrelia pathogenic species, 62 from Babesia microti, 15 from Bartonella henselae, 8 from Anaplasma phagocytophilum, 11 from Ehrlichia chafeensis, and 3 from Rickettsia parkeri and R. rickettsia for a total of 259 candidate biomarkers derived from multiple cell compartments (FIG. 10).

These biomarkers provided functional information about the metabolic and proliferative state of the pathogen that cannot be revealed by serology. The notion that urine is a viable fluid for detection of Borrelia derived biomolecules is corroborated by our previous publications^{1,3,7,8} and by independent literature⁹⁻¹¹.

The proposed concept (FIG. 8 and FIG. 9) is to seek independent validation of a panel of 160 urinary protein biomarkers collected under CAP/CLIA accreditation (7223012/49D2002076) that indicated clinical utility for the identification of Borreliosis in a published clinical study of 408 individuals under clinical evaluation for tick borne illnesses, compared to appropriate controls (FIG. 10).

In an embodiment, multiple reaction monitoring assays, selected and experimentally verified of specific conserved unique peptides for Borrelia species (B. afzelii, burgdorferi, bissettii, garinii, spielmanii, valaisiana, hermsii, parkeri, turicatae, hispanica, persica, crocidurae, miyamotoi), such as sequences specified in FIG. 9 (N=26), were chosen based on the following criteria: a) unique within the Borrelia species, with no overlap in a BLAST search against non-redundant protein sequences, and b) MRM acceptable peptides according to the criteria listed in 20-23 (e.g., absence of methionine and cysteine, absence of glutamine at the N terminus).

TABLE 1
Examples of Borrelia specific peptides identified
in the urine of patients affected by Borrelia infection.

| Peptide sequence | SEQ ID NO. | Borrelia species | Protein annotation |
|---|---|---|---|
| ININYAQEDDINK | SEQ ID NO. 1 | Borreliella afzelii | DNA-3-methyladenine |
| | | | glycosylase |
| FEINLDYVNEK | SEQ ID NO. 2 | Borrelia parkeri | ATP-dependent protease |
| MLLADEKDVIDSIR | SEQ ID NO. 3 | Borrelia turicatae | hypothetical protein |
| GGAQIIDPHDAKIISEI | SEQ ID NO. 4 | Borrelia duttonii | phosphomannomutase |
| QK | | | |
| MGLAQPVITQQMVIA | SEQ ID NO. 5 | Borrelia hermsii | BDR-repeat family |
| ELTR | | | protein |
| YFDNLSEEDVLKLNIP | SEQ ID NO. 6 | Borreliella | Crystal structure of a |
| TGIPLVYELDK | | burgdorferi | phosphoglycerate mutase |
| | | | gpmA |
| LGEVVAFFEYLNSVFL | SEQ ID NO. 7 | Borreliella | ribosome maturation |
| EVR | | burgdorferi | factor RimM |
| MITSIIEINK | SEQ ID NO. 8 | Borrelia crocidurae | Hypothetical protein |
| | | | BCD_0352 |
| DTDIFYYYRLDNK | SEQ ID NO. 9 | Borrelia hermsii | Putative laminin G |
| | | | domain-containing protein |
| GLGGIANIK | SEQ ID NO. 10 | Borrelia turicatae | PTS transporter subunit |
| | | | EIIC |
| GGNFVYSTLEFQVMQ | SEQ ID NO. 11 | Borrelia crocidurae | copper homeostasis |
| EDIEICK | | | protein CutC |
| VINLNIEAGHK | SEQ ID NO. 12 | Borrelia turicatae | hypothetical protein |
| NVVNAAYEVIK | SEQ ID NO. 13 | Borrelia duttonii | L-lactate dehydrogenase |
| RNVDNFDLSHLNWL | SEQ ID NO. 14 | Borrelia turicatae | transglycosylase |
| WNFDYAGK | | | |
| SVFMLAGEIK | SEQ ID NO. 15 | Borrelia persica | transcription |
| | | | termination/antitermination |
| | | | protein NusG |
| GLGSFDLQDK | SEQ ID NO. 16 | Borrelia turicatae | DNA gyrase subunit A |
| NMLDIISNNLNIIR | SEQ ID NO. 17 | Borrelia crocidurae | hypothetical protein |
| LDDIPGIIHSK | SEQ ID NO. 18 | Borrelia turicatae | hypothetical protein |
| | | | BT0238 |
| AVEESVVAMQSIVEK | SEQ ID NO. 19 | Borrelia parkeri | Methyl-accepting |
| | | | chemotaxis protein |
| NGVDGPLTKPLSPGG | SEQ ID NO. 20 | Borrelia duttonii | uncharacterized conserved |
| PQGVTNPGNGGGAV | | | protein |
| NDEQILK | | | |
| KMIAEYETLQQQIFK | SEQ ID NO. 21 | Borrelia duttonii | uncharacterized conserved |
| MQR | | | protein |
| RTDIASFQANIDITIDS | SEQ ID NO. 22 | Borrelia persica | hypothetical protein |
| LNSR | | | |
| SALTVGVGTIMDSQEI | SEQ ID NO. 23 | Borreliella | glucosamine-6-phosphate |
| LIIVNGHNK | | bavariensis | deaminase |
| FLSQQKDLEDK | SEQ ID NO. 24 | Borrelia duttonii | p-512 protein |
| INLYLVEGIDNK | SEQ ID NO. 25 | Borrelia turicatae | hypothetical protein |
| ISAIIMTLFMVLVSCN | SEQ ID NO. 26 | Borrelia hermsii | Variable outer membrane |
| SGGVAEDPK | | | protein |
| MDEMKSSFGILSNLL | SEQ ID NO. 27 | Borrelia crocidurae | hypothetical protein |
| MK | | | |
| NLYNTFLNIGTYFQMI | SEQ ID NO. 28 | Borrelia crocidurae | polyprenyl synthetase |
| DDIK | | | family protein |
| LENTAMPLVAEIGEV | SEQ ID NO. 29 | Borreliella | flagellar motor switch |
| K | | burgdorferiss | protein FliM |
| EIIPTINGELLDNIVDYI | SEQ ID NO. 30 | Borrelia parkeri | CDP-alcohol |
| NYTFIPTIFFYYGNFIS | | | phosphatidyltransferase |
| NEYK | | | family protein |
| IVTHNDLNGKNNSNS | SEQ ID NO. 31 | Borreliella | translation initiation factor |
| SINLDK | | bavariensis | IF-2 |
| NPIINEQQTLLTSIAW | SEQ ID NO. 32 | Borrelia turicatae | glycerol kinase |
| GR | | | |
| FAEFLLDTLFLNNK | SEQ ID NO. 33 | Borrelia duttonii | PF49 plasmid partition |
| | | | protein |
| LNENELFDVK | SEQ ID NO. 34 | Borrelia duttonii | nicotinate |
| | | | phosphoribosyltransferase |
| NEVTSITEDEQK | SEQ ID NO. 35 | Borrelia duttonii | hypothetical protein |
| | | | BDU_5017 |
| LPEVLYAYELDIR | SEQ ID NO. 36 | Borrelia recurrentis | YicC family protein |
| ENENETFQSEDNPNM | SEQ ID NO. 37 | Borrelia duttonii | chaperone protein GrpE |
| EK | | | |
| VETNGSSGVDNLIEEE | SEQ ID NO. 38 | Borrelia turicatae | fructose-specific PTS |
| EIER | | | transporter subunit EIIC |
| NLILINTNEFHNGIVL | SEQ ID NO. 39 | Borrelia crocidurae | 16S rRNA (uracil(1498)- |
| NDPR | | | N(3))-methyltransferase |
| NADISYYNQGIK | SEQ ID NO. 40 | Borrelia parkeri | Exodeoxyribonuclease V |
| | | | beta chain |
| GVTELEALNTAVDAL | SEQ ID NO. 41 | Borrelia hermsii | Variable small protein 1 |
| LK | | | |
| EKLLDEQIIQELSAIK | SEQ ID NO. 42 | Borrelia parkeri | polyprenyl synthetase |
| | | | family protein |
| IFLSENVSILNESLNIL | SEQ ID NO. 43 | Borrelia crocidurae | hypothetical protein |
| R | | | |
| QILAEFVDNNQLGYSI | SEQ ID NO. 44 | Borrelia hermsii | Sua5/YciO/YrdC/YwlC |
| NNR | | | family protein |
| INTYDIPQNNNLETED | SEQ ID NO. 45 | Borreliella | DNA mismatch repair |
| VNEPNK | | burgdorferiss | endonuclease MutL |
| LVRDSLLFYSSTNLAI | SEQ ID NO. 46 | Borrelia duttonii | single-stranded DNA- |
| LNFSIANNIK | | | binding protein |
| SIVGTMIDIEIK | SEQ ID NO. 47 | Borrelia hispanica | tRNA pseudouridine(38- |
| | | | 40) synthase TruA |
| YAELLEINK | SEQ ID NO. 48 | Borrelia turicatae | DUF1322 family protein |
| KDFNNNEIELR | SEQ ID NO. 49 | Borreliella afzelii | tyrosine--tRNA ligase |
| SLNIDITGDKVELR | SEQ ID NO. 50 | Borrelia turicatae | DUF685 domain- |
| | | | containing protein |
| IVDIDESNPNLPYINYII | SEQ ID NO. 51 | Borreliella | HD domain-containing |
| K | | burgdorferiss | protein |
| LFMSELSSIELSER | SEQ ID NO. 52 | Borrelia turicatae | DNA polymerase III |
| | | | subunit delta |
| LIGLQTEPNVIEFNDFL | SEQ ID NO. 53 | Borrelia turicatae | DUF685 domain- |
| NK | | | containing protein |
| TNPNEINALQTLNDY | SEQ ID NO. 54 | Borrelia turicatae | hypothetical protein |
| YYSNK | | | |
| IILGASGAEK | SEQ ID NO. 55 | Borrelia sp. | DNA translocase FtsK |
| NYLNEPSRDIITK | SEQ ID NO. 56 | Borreliella | hypothetical protein |
| | | burgdorferiss | |
| SLEVLGNEYTK | SEQ ID NO. 57 | Borreliella | oligoendopeptidase F |
| | | burgdorferiss | |
| ESISEQDIMQSSLNK | SEQ ID NO. 58 | Borrelia crocidurae | chromosome |
| | | | replication/partitioning |
| | | | protein |
| ENPNVLQIMQVEEDIK | SEQ ID NO. 59 | Borrelia persica | RelA/SpoT family protein |
| LGEASEELEKVAK | SEQ ID NO. 60 | Borrelia duttonii | vlp protein |
| LNCNLVFIDGKFVPK | SEQ ID NO. 61 | Borreliella afzelii | ribonuclease HII |
| DLLIVGLGNPGSNFFH | SEQ ID NO. 62 | Borrelia hermsii | aminoacyl-tRNA |
| TR | | | hydrolase |
| QDNLNFLSLEDLINLK | SEQ ID NO. 63 | Borrelia turicatae | hypothetical protein |
| MFLVMFFVSCVLFSE | SEQ ID NO. 64 | Borrelia turicatae | hypothetical membrane |
| EIYYR | | | associated protein |
| SGDIDLEVLK | SEQ ID NO. 65 | Borrelia hermsii | VWA domain-containing |
| | | | protein |
| ETGHENAYFPMLIPY | SEQ ID NO. 66 | Borreliella afzelii | proline--tRNA ligase |
| GFLEKEK | | | |
| ASEELEEVAKK | SEQ ID NO. 67 | Borrelia crocidurae | Variable major outer |
| | | | membrane lipoprotein |
| SGYIFEGINGVF | SEQ ID NO. 68 | Borrelia duttonii | hypothetical protein |
| | | | BDU_1019 |
| DKEFISQMAQFSALEQ | SEQ ID NO. 69 | Borrelia recurrentis | flagellar hook assembly |
| MTNMNK | | | protein FlgD |
| LSEIFEMIK | SEQ ID NO. 70 | Borrelia miyamotoi | cell division protein FtsA |
| IQGWMAESVGGEQVF | SEQ ID NO. 71 | Borrelia crocidurae | flagellar hook protein |
| NTSSDIR | | | FlgE |
| ISAALSDTGVTYSRFIE | SEQ ID NO. 72 | Borreliella | 50S ribosomal protein L20 |
| GLLK | | burgdorferiss | |
| TISYLNNIFNNEILRQE | SEQ ID NO. 73 | Borrelia turicatae | fructose-specific PTS |
| IMSTNDINR | | | transporter subunit EIIC |
| EIECAVIGNEQIK | SEQ ID NO. 74 | Borrelia parkeri | D-alanine--D-alanine |
| | | | ligase |

Proteins secreted from Borrelia in vivo provide important functional information about the pathogen metabolic weaknesses that can be targeted as new therapeutic approaches and can be pursued as future vaccine targets.

### Scientific Rational:

Borrelia shed peptides, similarly to cancer derived biomarkers, or molecules by other pathogens such as tuberculosis, can enter blood circulation by passively penetrating blood vessel walls¹³.

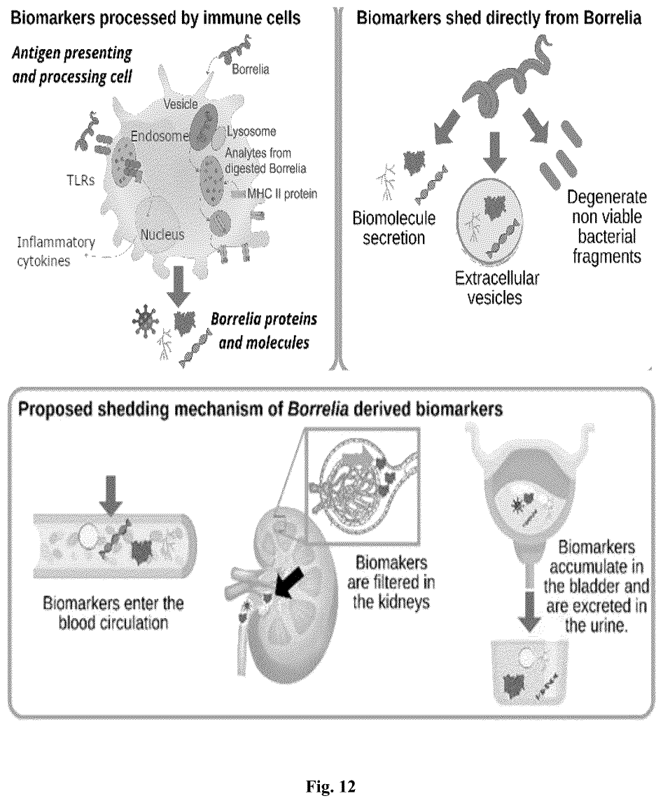

Blood circulating biomarkers undergo glomerular filtration and tubular reabsorption in the kidneys, are concentrated in the bladder, and eventually are excreted in the urine¹⁴ (FIG. 12).

In an embodiment, pathogen-shed biomarkers may reside in the blood for a short period of time, they are integrated over time in the urine¹³. Thus, urine appears to be Scientific rationale.

Borrelia shed peptides, similarly to cancer derived biomarkers or molecules shed, have advantages for Borreliosis diagnostics for several reasons. Urine integrates circulating low concentration analytes cleared in the kidneys over time, such that the total number of analyte molecules in the entire urine volume is much greater than those present in a spot blood sample. Urine testing is also non-invasive and can be easily conducted longitudinally following diagnosis and treatment of a patient presenting with a tick bite.

In an embodiment, we demonstrated feasibility of using urine for diagnosis of infection diseases such as Borreliosis³ and infectious diseases caused by other pathogens including Trypanosoma cruzi¹⁵, Mycobacterium tuberculosis^{4,12}, and Toxoplasma gondii¹⁶.

In an embodiment, product is a direct test of Borrelia shed sequence-specific proteins/peptides in urine, for application to early diagnosis. The banked patient cohort samples used for this study were collected under an IRB approved clinical study (GMU IRB approval number 869592). Mass spectrometry was performed in the applicant's CAP CLIA accredited laboratory, following CAP requirements for validation, proficiency, and quality assurance (Dr. Liotta Medical Director). Results are shared with treating physicians under the guidelines of the IRB approval, and the consent form specifying research use only. Physician input was solicited for defining Borrelia species to be included in the test. Patient feedback on the urine collection device ease of use was surveilled and used to modify the device design to include a handle. We will continue to utilize the banked Borreliosis samples from this trial to discover more specie-specific biomarkers identified in vivo. We will design and perform multiple reaction monitoring quantitative assays for the identified Borrelia peptides using independent, well characterized samples, to identify a diagnostic panel (10-20) of sensitive and specific biomarkers that accurately predict Borreliosis for individual marker cut point thresholds. We will then raise monoclonal antibodies and develop high throughput multiplexed antigen down immunoassays to measure the final set of biomarkers in the urine using our established high throughput multiplex immunoassay method¹⁷. We will correlate the outcome of the immunoassay panel cross-validated with MRM on a set of longidtudinally collected samples. Ultimately, if feasibility studies are successful, we will seek FDA guidance for possible regulatory approval of the immunoassay kit with the intended use of acute Borreliosis diagnostics.

We propose to leverage an existing collaboration with the Mason and Partners (MAP) Clinics, managed by Mason clinicians, who provide comprehensive primary and preventive health services to low-income populations, serving >600 patients per month.

Using the verified immunoassays, we will perform a surveillance study on 10,000 individuals, with populated Borreliosis levels, to establish prevalence of emphasis on underserved populations, outdoor workers and dark skinned individuals, who are at higher risk of disease, and, because of their dark skin tone, can mask a tick bite rash, and who may have a higher risk of late diagnosis, delayed treatment and progression disseminated disease^{18,19}.

We established, clinically validated, and published a Borreliosis and tick-borne illness assay that achieves high diagnostic sensitivity and specificity. We have applied the unbiased proteomics approach to identify tick borne pathogen peptides in the urine of patients under consideration for tick borne illnesses at different stages. The assay has an analytical sensitivity of 2.5 pg/mL³.

In a cohort of 408 cases and controls (Table 2), we have identified 2 pathogen derived peptides in 12/13 acute EM cases, and 0 false negatives in 250 asymptomatic and symptomatic controls (data partially published in Magni et al³ and Table 3). We found that 40% of PTLDS patients and patients under clinical evaluation for tick borne illnesses had urinary peptides derived from a tick borne pathogen³. Targeted pathogens were Borrelia, Babesia, Anaplasma, Rickettsia, Ehrlichia, Bartonella, Francisella, Powassan virus, tick-borne encephalitis virus, and Colorado tick fever virus.

TABLE 2
Patients Characteristic

| | | | | Diseased | Healthy |
|---|---|---|---|---|---|
| Characteristics | Acute LB | PTLDS | NA-TBI | Controls | Controls |
| N | 13 | 36 | 112 | 215 | 35 |
| Age (Median) | 52.5 | 50 | 46 | 35.5 | 31 |
| | (IQR = 14) | (IQR = 25) | (IQR = 32) | (IQR = 31) | (IQR = 60) |
| Sex | | | | | |
| Male/Female | 6/7 | 14/22 | 54/58 | 112/57 | 18/17 |
| NA | — | — | — | 46 | — |
| Tick | 8Y/1N | 11Y/10N | 11Y/15N | — | — |
| discovered | | | | | |
| Erythema | 13Y/0N | 26Y/5N* | 24N* | — | — |
| migrans rash | | | | | |
| Muscokeletal | 10Y/0N | 22Y/4N | 15Y/N | — | — |
| Pain | | | | | |
| Fatigue | 10Y/0N | 18Y/6N | 20Y/4N | — | — |
| Fever | 10Y/0N | 11Y/13* | 7Y/17N* | — | — |
| Facial Palsy | 1Y/6N | 3Y/20N* | 3Y/21N* | — | — |
| Cognitive | 4Y/0N | 16Y/7N | 9Y/14N | — | — |
| Impairment | 4Y/0N | 16Y/7N | 9Y/14N | — | — |
| Serology | 13Y/0N** | 19Y/4N | 12Y/14N | | |
| (Pos) | | | | | |

TABLE 3
Borrelia peptides identified in acute stage Borreliosis patients.

| | | | | Under | Number of | |
|---|---|---|---|---|---|---|
| | | Acute | | treatment | identified | |
| Sample | EM | LB | | (at time of | Borrelia | |
| ID | Rash | symptoms | Serology | collection) | peptides | Peptide sequence |
| 108838 | Y | Y | Pos | No | 3 | IDTEEAAVK; NAGNFEIIR; |
| | | | | | | VTLSDISTK |
| 104821 | Y | Y | Pos | No | 2 | AILKTNGDKTLGAAELEK; |
| | | | | | | NNFCLSKSDLEEIR |
| 790907 | Y | Y | Pos | No | 2 | SNQDNQTLLLSLHQALAK; |
| | | | | | | LKNSHAELGVAGNGATTDENAQK |
| 889597 | Y | Y | Pos | Yes | 2 | GGYLDHVNHAGAKKVILTVPAK |
| | | | | (2 days | | LATVNEAEGK |
| | | | | doxycycline) | | |
| 213567 | Y | Y | Pos | No | 2 | LATVNEAEGK; NDVSEEKPEIK |
| 453742 | Y* | Y* | Pos | No | 3 | VVILNEATGK; LATVNEAEGK; |
| | | | | | | FVYIGNVDNMGYTINFK |
| 463256 | Y | N | Pos | No | 2 | NLSLFTDFYEISMMNAYFIK; |
| | | | | | | QKATGAINAVSGEQIL |
| 459235 | Y | Y | Pos | Yes | 2 | SAKEVINNTSTMEK; |
| | | | | (1 day | | SSSVDGFVSFKEYKER |
| | | | | doxycycline) | | |
| 729340 | Y | Y | Pos | No | 1 | FEDAIVLRDK |
| 310741 | Y | Y | Pos | No | 2 | VNESDLGIKALDEK; |
| | | | | | | FNVEACFPTLIVEK |
| 111039 | Y | Y | Pos | No | 3 | KTNKIVFIFGPTAVGK; |
| | | | | | | MRDIIAISSIKGGVGK; |
| | | | | | | DLKVKHIGIR |
| 111101 | Y | Y | Pos | No | 8 | YFDNLSEEDVLKLNIPTGIPLVYELDK; |
| | | | | | | AFDVLHQGLKPYHHADNK; |
| | | | | | | NILILMIPNK; LDDIPGIIHSK; |
| | | | | | | EKDLEERLINEQK; |
| | | | | | | DHLALMQDSQDVDGINNLGNK; |
| | | | | | | TLNELINMLDNLYEK; |
| | | | | | | MLEFETIDINLTK |

* developed after collection

### Acute Borreliosis.

All 13 acute patients were later verified to be two tier serology positive 3 to 6 weeks later (CDC criteria) (Table 2). The control group consisted of 100 patients (M/F=1, median age=41, IQR=25.75), including healthy controls and disease control patients diagnosed with acute respiratory distress syndrome, tuberculosis, and traumatic brain injury (Table 3). Peptides derived from Borrelia species known to be pathogenic in humans were found in 13/13 Borreliosis patients, and two or more peptides were found in 12/13 samples. No peptides derived from other tick-borne organisms investigated were identified.

### Late Stage Borreliosis and Other Tickborne Illnesses.

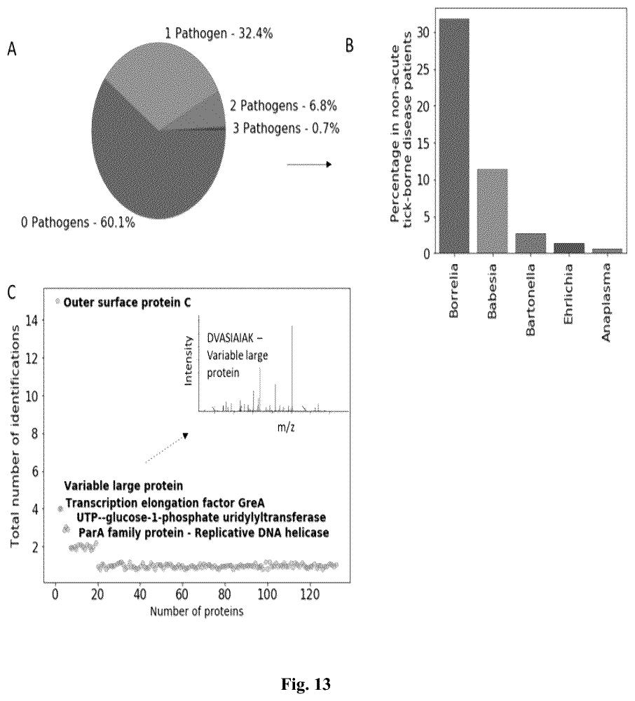

59/148 non acute patients (40%) were positive for at least one tick-borne pathogen and all the controls were negative. n=48 were positive for Borrelia, n=17 positive for Babesia, n=4 were positive for Bartonella, n=2 were positive for Ehrlichia, n=8 were positive for Borrelia and Babesia, n=1 was positive for Borrelia and Bartonella, n=1 was positive for Babesia and Bartonella, n=1 was positive for Babesia, Bartonella and Anaplasma. Therefore, 48/148 patients (32%, FIG. 13) were positive for only one pathogen and 10/148 samples (7%) were positive for 2 pathogens, and 1/148 (0.7%) was positive for 3 pathogens suggesting the co-existence of multiple infections (FIG. 13).

In an embodiment, urine peptidomics provides clues to functional weaknesses of Borrelia related to carbohydrate metabolism, phospholipid metabolism, fatty acid metabolism, and oxidative stress that can be targeted as new therapeutic approaches (FIG. 10).

Concerning biomarker collection and preservation technology for urine sample shipping at room temperature, our affinity network technology can be incorporated in a novel “origami” collection envelope that folds 100 mL of urine fluid biomarkers into a dry confidential envelope for secure mail service transport, completely obviating the need for liquid or frozen urine handling shipment or storage (FIG. 11).

All patient samples have been collected under full consent. All discovery, verification, blinded validation, and independent cross validation between Labs will follow College of American Pathologists (CAP)/Clinical Laboratory Improvement Amendments (CLIA) guidelines under our accreditation: CAP 7223012 CLIA 49D2002076. This includes formalized authentication of reagents, proficiency testing, blinded assay verification, sensitivity, lack of carry-over, quality assurance, data storage, patient confidentiality, and sample, collection, handling, and storage at-80C. All urine samples undergo full urinalysis including specific gravity, leukocytes, nitrites, glucose, hemoglobin, creatinine, ketones, and bilirubin.

### Materials

Prepare all solutions with 18 MΩ-cm water and analytical grade reagents. Prepare and store all reagents at room temperature unless noted otherwise. Follow waste disposal regulations when disposing of waste materials.

### 2.1 Affinity Net Preparation

1. Twisted Nylon Seine Twine (diameter 1.07 mm, length 2268 ft) (see Note 1).
2. Dyes: Sudan IV, Sudan Black B.
3. Dispersing agents: Alconox, sodium 1-naphthalenesulfonate.
4. Dye bath: a solution of glacial acetic acid in water at a pH range of 4 to 5.5.
5. Wash solution 1: dissolve 0.1 g of Alconox powder soap in 500 mL of water.
6. Wash solution 2: mix 1 mL of Triton X-100 with 500 ml of water.
7. 100 um cell strainers.
8. Ice.
9. 15 mL conical tubes.
10. 50 ml conical tubes.
11. Convection oven that reaches 130 degrees Celsius.
12. pH meter.
13. Precision scale with readability≤0.001 g and linearity≤0.002 g.
14. Vortex Genie Lab mixer.
15. Heat resistant gloves.
16. Tube rotator.
17. Chemical hood.
   2.2 Affinity net validation
1. Dyed nylon prepared in 2.1 Affinity net preparation.
2. Nylon dissolving solution: dissolve CaCl₂) to a concentration of 22% w/v in methanol (11 g of CaCl₂) in a total volume of 50 mL methanol).
3. Dyes: Sudan IV, Sudan Black B.
4. 96-Well UV Plates with Flat Bottom (370 uL volume).
5. UV-Vis microplate absorbance reader.
6. 96 well plate sealing tape.
7. Hand carder for fiber combing.
8. Wash solution: 1× phosphate buffer saline (PBS).
9. 100 μm cell strainers.
10. Extraction buffer: Laemmli buffer with 5% beta mercaptoethanol.
11. Precast 4-20% gradient Tris-glycine gel.
12. Electrophoresis apparatus for SDS PAGE.
13. Tris-glycine running buffer.
14. Protein gel silver staining kit.
15. Microcentrifuge.

### 2.3 Urine Sample Incubation

1. Multistix 10 SG Urine Reagent Strips.
2. Urine negative control: 40 mL of urine collected from a healthy human subject.
3. Urine positive control: spike 1 μL of Borrelia burgdorferi lysate (Creative Biomart) in 40 mL of healthy donor urine.
4. Recombinant chicken lysozyme.
5. 1M hydrochloric acid and 1M sodium hydroxide to adjust the pH of urine if needed.
6. Affinity net.
7. Elution Buffer: 1 mg of RapiGest (Waters Corporation), 190 μL of 50 mM Ammonium bicarbonate, 10 μL of tris (2-carboxyethyl) phosphine (TCEP, Thermo Scientific). Once the elution buffer is prepared, it should be used immediately. Unused elution buffer should be quickly refrigerated and stored at 4° C. for 1 week maximum.
8. Alkylating solution: 500 mM iodacetamide prepared by dissolving 18.5 mg of iodacetamide in 200 μL of 50 mM ammonium bicarbonate.
9. Trypsin solution: mix 40 μL of Trypsin Resuspension Dilution Buffer to a vial containing 20 μg of lyophilized Sequencing Grade Modified Trypsin (Promega).
10. Trifluoracetic acid.
11. Pierce C18 Spin Columns.
12. C18 spin column activation Solution: 0.1% formic acid in 80% acetonitrile.
13. C18 spin column wash Solution: 0.1% formic acid.
14. C18 spin column equilibration solution: 0.1% formic acid.
15. C18 spin column elution buffer: 0.1% formic acid in 80% acetonitrile.
16. 1.5 mL microcentrifuge tubes.
17. 15 mL centrifuge tubes.
18. pH strips.
19. Benchtop Centrifuge.
20. Microcentrifuge.
21. Orbital Shaker.
22. 24 Position MICROVAP Nitrogen Manifold.

### 2.4 Mass Spectrometry Analysis

1. Thermo Scientific Orbitrap Exploris 480.
2. Nanospray EASY-nLC 1200 UHPLC.
3. Acclaim™ PepMap™ C18 HPLC Columns.
4. Mobile phase A: 0.1% formic acid in ultrapure water for UHPLC, suitable for mass spectrometry (e.g., Sigma 900682); and mobile phase B: 0.1% formic acid and 80% acetonitrile in ultrapure water.
5. Proteome Discoverer or another proteomic analysis software.

### 3. Methods

### 3.1 Affinity Net Preparation (Nylon Dyeing)

1. Weight 0.5 g of nylon.
2. Weight 0.15 g of dye (Sudan Black B or Sudan IV).
3. Weight 0.02 g of a dispersing agent (see Note 2).
4. Prepare 5 mL of dye bath, which is a solution of acetic acid in water at a pH range of 4-5.5 (see Note 3).
5. Place the nylon into a 15 ml conical tube and add 5 ml of the dye bath.
6. Place the conical tube containing the dye bath and the nylon samples into a convection oven and let the temperature reach 60° C. This will allow the fibers to expand.
7. Once the oven reaches 60° C., incorporate the dye and the dispersing agent. Vortex until full dissolution (see Note 4).
8. Increase the oven temperature to 130° C. (should take about 30-40 min) and hold for 1 hr. Agitation of samples every 15-20 min is recommended to increase the diffusion-adsorption of the dye onto the fibers. In order to mix the solution, remove the conical tube from the oven using heat resistant gloves and vortex the conical tube vigorously five times [Shang, S. M. (2013). Process Control in Textile Manufacturing∥Process control in dyeing of textiles. ( ), 300-338. doi: 10.1533/9780857095633.3.300]. Immediately place the conical tube in the oven.
9. Next, turn off the oven and let the samples cool down slowly. This step should take approximately 40 minutes.
10. Once at room temperature, place the tubes into an ice bath for 15 min.
11. Using a sterile 100 um cell strainer, discard the liquid and retain the nylon. Place the nylon sample in a new 50 mL conical tube.
12. Prepare wash solution 1 by dissolving 0.1 g of Alconox powder soap in 500 mL of water
13. Wash the nylon samples with 40 mL of wash solution 1 for 15 minutes in the rotator, discard washing solution and repeat until no free dye is observed (usually 6 times).
14. Prepare wash solution 2 (0.02% Triton X-100) by dissolving 1 ml of Triton X in 500 ml of water.
15. Wash the nylon samples with 40 mL of wash solution 2 for 15 min in the rotator once. Discard wash solution 2.
16. Wash the nylon samples with 40 mL of water for 15 min in the rotator to eliminate the detergent. Discard wash water and repeat until detergent is removed. Verify complete absence of bubbles in the solution upon vortexing before proceeding to the next step.
17. Discard water and place the nylon into a new conical tube, leave the tube open and allow the nylon to dry overnight under the chemical hood.

### 3.2 Affinity Net Validation

### Estimation of the Quantity of Bound Dye.

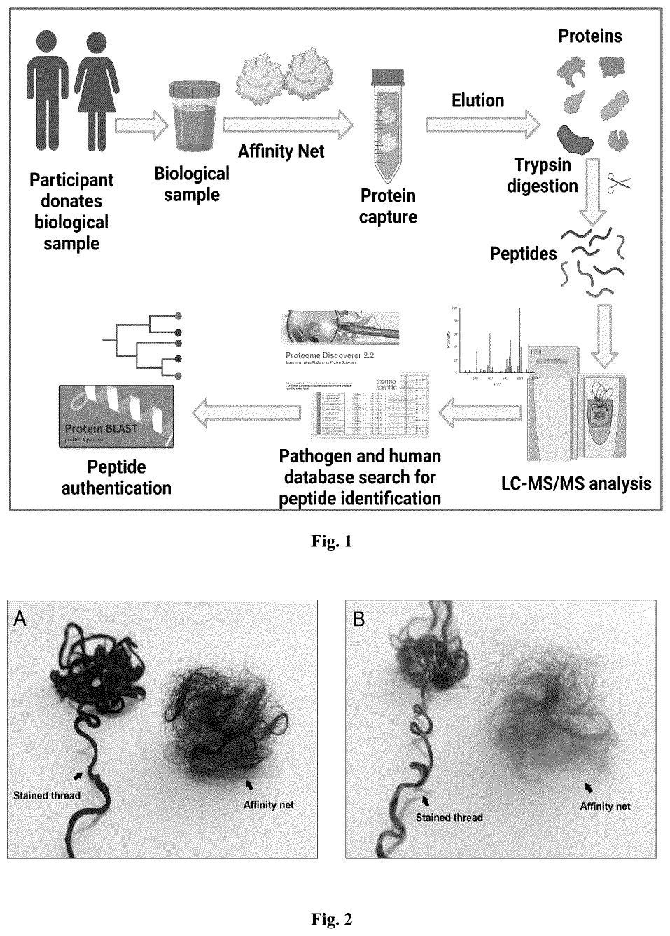

1. Perform UV-Vis spectroscopy to determine the amount of dye incorporated in the nylon fibers. Cut 6 mg of dyed nylon, place it into a 2 ml Eppendorf tube, and dissolve the sample adding 1000 ul of a solution of 22% CaCl₂/MeOH (see Note 5).
2. Place the tubes in the rotator at room temperature for 2 hrs or until full fiber dissolution by visual inspection. Vortex to promote fiber dissociation during incubation.
3. Prepare Sudan Black B and Sudan IV calibration curves. Prepare a stock solution weighing 10 mg of dye and dissolve it in 10 mL of 22% w/v CaCl₂)/MeOH.
4. In a 96-well plate, prepare seven two-fold serial dilutions for each dye using the 22% CaCl₂)/MeOH solution as diluent (total volume per well=150 μL). Sealing tape for the 96-well plate is recommended to avoid evaporation of methanol.
5. Place the 96-well plate into a microplate reader. Perform a spectral scanning between 300-700 nm (in 5 nm increments) to identify the optimal wavelength of the dye absorption.
6. Using the optimal wavelength identified in step 22, measure the absorbance of the calibrators, and build the standard curve.
7. Transfer 150 μl of the dissolved dyed nylon obtained in step 19 into the 96-well plate and measure the absorbance using the optimal wavelength identified in step 22.
8. Calculate the ratio of bound dye/nylon (% W/W). Expected ratios range between 0.5-2%.
9. Using a sterilized carder, unthread and comb nylon threads until individual fibers are separated (FIG. 2). The combed, dyed nylon constitutes the affinity net (standard unit=1 cm, 20 mg).
10. Autoclave the affinity net at 121° C. for 15 minutes.

### Protein Capture

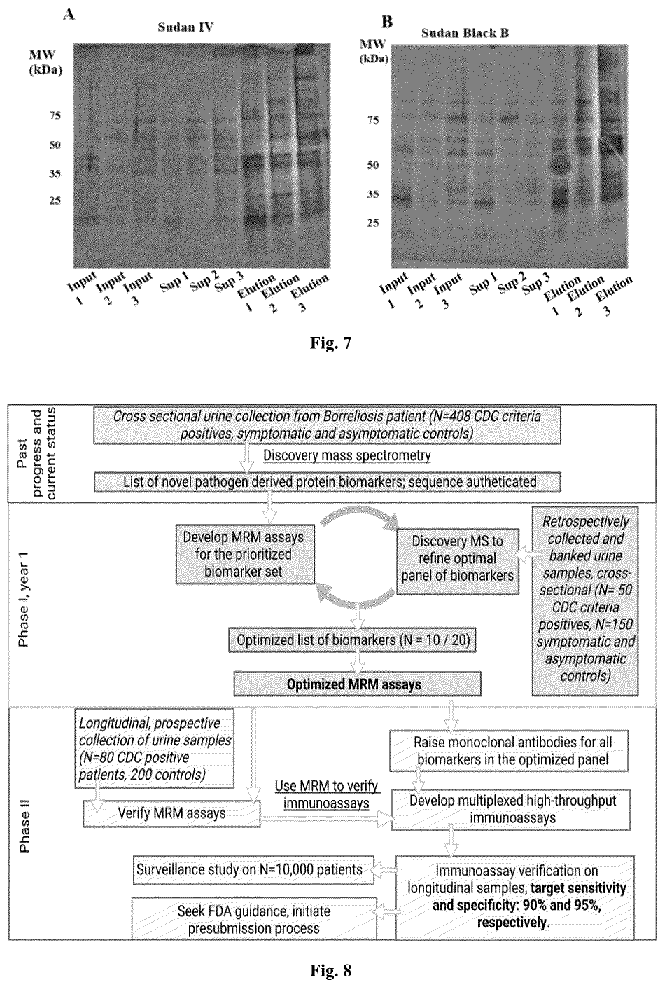

1. Each batch of affinity net is validated using human urine and SDS PAGE analysis. Under informed consent, obtain healthy volunteer control urine. Centrifuge aliquots of 11 mL of urine at 3,700×g for 15 minutes. Transfer 10 mL of clarified urine in a 50 ml conical tube, 20 μL of clarified urine in a 1.5 mL Eppendorf tube (initial solution) and discard the urine debris.
2. Using sterile tweezers, add affinity net (20 mg) to the urine in the 50 mL conical tube.
3. Let the affinity net incubate with urine for 30 minutes at room temperature under rotation.
4. After incubation, collect 20 μL of unbound urine liquid with a pipette and place it in a 1.5 mL Eppendorf tube (supernatant), discard the remaining liquid through a 100 μm cell strainer, and place the retained affinity net in a new labelled Eppendorf tube using sterile tweezers.
5. Wash the affinity with 1 mL of 1×PBS. Centrifuge the tube at 16.1 rcf for 5 minutes at room temperature and remove the PBS using a 1000 μL pipette.
6. Add 100 μL of Laemmli sample buffer additioned with 5% beta mercaptoethanol to the affinity net and let incubate for 10 minutes at 95° C.
7. Centrifuge the affinity net tube at 16.1 rcf for 5 minutes at room temperature. Save the supernatant (eluate) in a new Eppendorf tube.
8. Add 20 μL of Laemmli sample buffer additioned with 5% beta mercaptoethanol to the initial solution of step 1, and to the supernatant of step 4 and let incubate for 10 minutes at 95° C.
9. Load 30 μL of initial solution, supernatant and eluate in a 4-20% Tris Glycine SDS PAGE gel.
10. Separate proteins for 1 hour at 150 V and at room temperature.
11. Stain the gel using Silver Stain kit according to vendor's instructions. A representative gel is shown in FIG. 7, where the eluate has higher protein content and different protein pattern than the initial solution, where bands absent in the initial solution appearing in the eluates.

### 3.3 Urine Sample Processing

1. Seek approval or exemption to conduct human subject research from your Institutional Review Board (IRB). For this analysis, spot urine samples are collected from donors, who signed an informed consent, in sterile 100 mL urine cups without additives. Store urine samples at −80° C. When ready to perform the analysis, thaw the frozen urine samples by placing the tightly sealed urine cups in warm water (approximately 37° C.). Change the water every 5 minutes until samples are completely thawed. Procure 40 mL of normal, healthy urine to serve as a negative control, and another 40 mL for a positive control. The positive control is obtained by spiking 1 μL of Borrelia cell lysate into 40 mL of healthy urine.
2. Using a Multistix 10 SG reagent strip, perform urinalysis on 1 mL urine aliquots according to manufacturer instructions. Note any measurements that are not within the acceptable ranges.
3. Centrifuge all 40 mL urine samples at 3,700×g for 15 minutes.
4. Remove urine from centrifuge and decant supernatant into a new tube. Discard pellet.
5. If the urine pH is different than 5.5, adjust the pH 5.5 (see Note 6). If the pH is above 5.5, then incrementally add 200 μL of 1M hydrochloric acid until pH 5.5 is reached. When adjusting the pH, measure the urine pH with pH paper. If the urine pH is below 5.5, then incrementally add 200 μL of 1M sodium hydroxide until pH 5.5 is reached.
6. Spike 12 ng of chicken Lysozyme in each sample. Chicken lysozyme is a process control that is monitored in the final mass spectrometry analysis.
7. Using sterilized tweezers, add the affinity net into each urine sample. Mix by inverting the tubes. Allow the affinity net to harvest proteins for at least 30 minutes at room temperature under rotation.
8. Discard the unbound liquid through a 100 μm cell strainer, and place retained affinity net in a new labelled Eppendorf tube using sterile tweezers.
9. Wash the affinity net with 1 mL of 1×PBS. Centrifuge the tubes at 16.1 rcf for 5 minutes at room temperature. Discard the supernatant.
10. Add 40 μL of Elution Buffer, containing RapiGest (see Note 7), 50 mM Ammonium bicarbonate, and TCEP, to the affinity nets and allow the affinity nets to incubate for 40 minutes at room temperature.
11. Centrifuge the samples at 16,100×g for 10 minutes.
12. Remove and save the supernatant (eluates) to new microcentrifuge tubes.
13. Prepare the alkylating solution 500 mM iodoacetamide (18.5 mg iodoacetamide in 200 ul of ammonium bicarbonate 50 mM) and keep it in the dark.
14. Add 160 μl of 50 mM ammonium bicarbonate to the eluates and invert few times.
15. Add 13 μl of alkylating solution to the eluates and invert a few times.
16. Quickly spin the samples and incubate in the dark for 20 minutes.
17. Add 4 μl of Trypsin (0.5 μg/μL) and let the samples incubate overnight at 37° C.
18. (The following day) add 2 μL of TFA to the eluates and mix well to stop trypsin digestion. Incubate for 30 minutes at 37° C. to degrade the Rapigest acid labile detergent bound to proteins.
19. Use Pierce C18 Spin Columns to clean up peptide solutions according to manufacturer instructions. Tap column to settle resin. Remove the top and bottom cap. Place columns into a receiver tube.
20. Add 200 μL of Activation Solution (0.1% formic acid, 80% acetonitrile) to rinse the walls of the spin column and to wet the resin.
21. Centrifuge the resins at 1500×g for 1 minute. Discard flow-through.
22. Repeat steps 2 and 3.
23. Add 200 μL Equilibration Solution to the resins. Centrifuge at 1500×g for 1 minute. Discard the flow-through.
24. Repeat step 5.
25. Place the columns into labeled 2 mL tubes.
26. Load the digested samples of step 18 on top of the resin bed. Centrifuge at 1500×g for 1 minute.
27. To ensure complete binding, recover flow-through and repeat step 8.
28. Discard the flow-through.
29. Add 200 μL of Wash Solution to the columns and centrifuge at 1500×g for 1 minute. Discard the flow-through.
30. Repeat step 11.
31. Place columns into new labeled 1.5 mL tubes.
32. Add 20 μL of Elution Buffer to the top of the resin bed.
33. Centrifuge at 1500×g for 1 minute.
34. Repeat steps 33 and 34 for each tube.
35. Gently dry the samples under nitrogen flow using a nitrogen manifold.
36. Store dried samples at-20° C.

### 3.4 Mass Spectrometry Analysis

1. Reconstitute the digested samples in 10 μL of 0.1% Formic Acid in water.
2. Perform LC-MS/MS on an Exploris 480 (Thermo Scientific) coupled with a nanospray EASY-nLC 1200 UHPLC. Perform Reversed-phase chromatography separation of the peptide mixture using PepMap RSLC 75 μm i.d.×15 cm long with 2 μm, C18 resin LC column (ThermoFisher). Use 0.1% formic acid as mobile phase A, and 0.1% formic acid, 80% acetonitrile as mobile phase B.
3. Elute peptides using a linear gradient of 5% mobile phase B to 50% mobile phase B in 90 min at 300 nL/min, then to 100% mobile phase B for an additional 2 min.
4. Operate the Exploris 480 in a data-dependent mode in which each full MS scan is followed by TopN MS/MS scans of the most abundant molecular ions with charge states from 2+ to 4+ that are dynamically selected for higher-energy collisional dissociation (HCD) using a normalized collision energy of 35%. Search tandem mass spectra where searched against microorganism databases with Proteome Discoverer 2.1 software using tryptic cleavage constraints.
5. Download databases for the following microorganisms from NCBI, UniProt, and PiroplasmaDB: Borrelia burgdorferi sensu strictu, B. chiliensis, B. bissetii, B. afzelii, B. garinii, B. spielmani, B. bavariensis, B. lusitaniae, B. valaisiana, B. mayonii, B. miyamotoi, B. hermsii, B. duttonii, B. turicatae, B. parkeri, B. hispanica, B. persica, B. caucasica, and B. crocidurae (See Notes 8 and 9).

### 3.5 Peptide Authentication

1. Statistical and physical parameters for spectrum matching to use in Proteome Discoverer are as follows: 1) Peptide false discovery rate (FDR) based on forward-reverse decoy <1%. 2) Xcorr>2.0, >3.0 and >4.0 for 2+, 3+, 4+ precursor ions. 3) q-value <0.05. 4) precursor ion mass tolerance <2 ppm and fragment ion mass tolerance <0.5 Da. 5) If the precursor is triply charged: 5.1) Presence of a basic residue (K, R, H) within the sequence (excluding N-terminus and C-terminus residues), and 5.2) Presence of corresponding doubly charged precursor ion in the full mass chromatogram (MS1).
2. Identify peptides that are unambiguously attributed to pathogenic Borrelia species. Discard peptide sequences shorter than 7 amino acids in order to minimize random error of attribution when searching for short-peptide sequences. Perform taxonomic assignment using a bioinformatics tool that identifies the lower unambiguous taxonomic rank for each peptide. An example algorithm is Unipept [doi: 10.1021/acs.jproteome.8b00716, PMID: 26819472] which uses the Lowest Common Ancestor (LCA) algorithm to generate taxonomic profiles of peptides. Unipept lists species and genus-unique peptides, which are considered discriminative peptides. Only discriminative peptides are considered for the identification of Borrelia peptides in bodily fluids.
3. Validate protein database annotation. Align the full-length protein, to which every peptide was attributed, with homologous proteins of evolutionary related organisms in the clade. Verify that the full-length protein had greater than 60% identity with proteins in the query. If the 60% identity threshold is reached, the database annotation is considered valid. An example workflow is as follows: For Borrelia, consider the following species: Borrelia burgdorferi, Borrelia garinii, Borrelia afzelii, Borrelia bavariensis, Borrelia mayonii, Borrelia miyamotoi, Borrelia hermsii, Borrelia turicatae, Borrelia recurrentis, Borrelia chilensis, Borrelia crocidurae, Borrelia duttonii, Borrelia bissettii. Align full length homologous proteins in these selected organism species using the JalView software. Verify the identity percentage.
4. Based on previous clinical studies [Magni et al], a urine sample is considered positive for Borrelia if two or more discriminative peptides are identified, and suspicious if one discriminative peptide is found (Table 1).

### 4. Notes

1. The nylon fibers used in this manuscript were purchased from SGT knots. Nylon fibers can be sourced by many companies. The purity of commercial polyamide 6 (nylon) fibers is very important for the overall yield of protein capture. Contaminants or coatings can hinder dye binding and can interact with bio-analytes in a non-specific and uncontrollable way. It is recommended that nylon fibers are authenticated via Fourier-transform infrared spectroscopy (e.g., Agilent Cary 630 FTIR spectrometer). Scans between the wavelengths 700 and 3600 should be compared with standard libraries for peak consistency (e.g., Hummel polymer sample library polyamide 6+polyamide 6.6). For this protocol, nylon was purchased in the form of threads and was unwound before starting the dyeing process.
2. Suggested dispersing agents include Alconox and sodium 1-naphthalenesulfonate. Use the reagents as received from the vendor without further purification.
3. To prepare the dye bath, start by adding 50 μL of glacial acetic acid to 1 liter of MilliQ water. Then fill a 250 mL container containing a magnetic stirrer with 200 mL of water, add a few drops of the diluted acetic acid solution with a plastic pipette, while stirring and monitoring the pH with a pH meter. Keep adding drops until you reach the target pH range of 4-5.5. If the pH drops to values lower than 4, discard the solution and start again. The pH cannot be titrated with a base.
4. The glass transition temperature (Tg) of nylon is 50-60° C. At this temperature, the fibers swell. (Shang, S. M. (2013). Process Control in Textile Manufacturing∥Process control in dyeing of textiles., ( ), 300-338. doi: 10.1533/9780857095633.3.300) 5. Always operate under the chemical hood when mixing CaCl₂) with CH₃OH. Pour 20 mL of CH₃OH into a 50 mL conical tube, then add 11 g of CaCl₂) in small quantities of approximately 1 gram at a time. Vortex the solution until the powder is completely dissolved. The temperature of the solution will increase, because the chemicals create an exothermic reaction. Once 11 g of CaCl₂) are dissolved in 20 mL of CH₃OH, add more CH₃OH until the total volume is 50 mL [doi.org/10.1295/polymj.27.631].
6. The urine pH is important because it influences protein capture, it affects the protonation of the dyes and biomolecules present in the urine. The optimal pH value of 5.5 has been established [Magni].
7. Rapigest is an acid labile detergent commonly used in mass spectrometry sample preparation because of its favorable performance yielding high peptide recovery and low background signal. An alternative approach can employ 4% sodium dodecyl sulfate (SDS) in 50 mM ammonium bicarbonate [Magni et al] instead of Rapigest. If using SDS, Thermo Scientific HiPPR and Pierce detergent removal resins should be added to the protocol to minimize SDS content before trypsin digestion and mass spectrometry analysis.
8. To maximize specificity, establish a laboratory contaminant database, which is composed of recurring contaminant peptides, including keratins [doi.org/10.1016/j.jprot.2013.07.009].
9. The current approach can be extended to other tick-borne pathogens using protein databases for Babesia microti, Francisella tularensis, Ehrlichia chaffeensis, Rickettsia rickettsiae, Rickettsia parkeri, Rickettsia species 364D, Rickettsia akari, Anaplasma phagocytophilum, Bartonella henselae, Powassan virus, Tick-borne encephalitis virus, Colorado tick fever virus. In order to analyze other tick-borne pathogens, the method will need to be calibrated by analyzing microorganism lysate spiked in healthy donor urine to establish the analytical measurement range. A contaminant database will be obtained by running healthy donor urine samples and analyzing samples with each pathogen protein database [doi.org/10.1002/pmic.201000103].

### INCORPORATION BY REFERENCE

All references, issued patents and patent applications cited within the body of the instant specification are hereby incorporated by reference in their entirety, for all purposes.
1: PMID: 26537892; 2: PMID: 30391228; 3: PMID: 33168903; 4: PMID: 29237757; 5: PMID: 32793606; 6: PMID: 33537276; 7: PMID: 21035184; 8: PMID: 32435898; 9: PMID: 8923044; 10: PMID: 15206688; 11: PMID: 17225014; 12: PMID: 31474995; 13: PMID: 30248975; 14: PMID: 29579412; 15: PMID: 25275534; 16: PMID: 33651824; 17: PMID: 34620701; 18: PMID: 34595684; 20: PMID: 22669653; 21: PMID: 19596694; 22: PMID: 17389395; 23: PMID: 16332733; 24: PMID: 21999289; 25: PMID: 33267867; 26: PMID: 22639787; 27: CDRH, F., USDHHS (2007).
(1) Pauwels, J.; Gevaert, K. Mass Spectrometry-Based Clinical Proteomics-a Revival. Expert Rev. Proteomics 2021, 18 (6), 411-414.
(2) Coipan, C. E.; van Duijvendijk, G. L. A.; Hofmeester, T. R.; Takumi, K.; Sprong, H. The Genetic Diversity of Borrelia afzelii Is Not Maintained by the Diversity of the Rodent Hosts. Parasit. Vectors 2018, 11 (1), 454.
(3) Fang, X.; Zhang, W.-W. Affinity Separation and Enrichment Methods in Proteomic Analysis. J. Proteomics 2008, 71 (3), 284-303.
(4) Kim, B.; Araujo, R.; Howard, M.; Magni, R.; Liotta, L. A.; Luchini, A. Affinity Enrichment for Mass Spectrometry: Improving the Yield of Low Abundance Biomarkers. Expert Rev. Proteomics 2018, 15 (4), 353-366.
(5) Magni, R.; Espina, B. H.; Liotta, L. A.; Luchini, A.; Espina, V. Hydrogel Nanoparticle Harvesting of Plasma or Urine for Detecting Low Abundance Proteins. J. Vis. Exp. JoVE 2014, No. 90, 51789.
(6) Yu, F.; Qiu, F.; Meza, J. 12-Design and Statistical Analysis of Mass-Spectrometry-Based Quantitative Proteomics Data. In Proteomic Profiling and Analytical Chemistry (Second Edition); Ciborowski, P., Silberring, J., Eds.; Elsevier: Boston, 2016; pp 211-237.
(7) Issa Isaac, N.; Philippe, D.; Nicholas, A.; Raoult, D.; Eric, C. Metaproteomics of the Human Gut Microbiota: Challenges and Contributions to Other OMICS. Clin. Mass Spectrom. Mar Calif 2019, 14 Pt A, 18-30.

What is claimed is:

1. A method for diagnosing Lyme disease, comprising:
isolating a diagnostic composition from a biological sample, wherein the biological sample is urine or another bodily fluid, the diagnostic composition comprising a biomarker attributable to Borrelia strains, bound or complexed to an affinity molecule that is bound or complexed to a filamentous material; and
analyzing isolated diagnostic composition using a mass spectrometry, an immunoassay, a protein sequencing, or other analytical methods to detect presence of Borrelia-derived biomarkers.

2. The method according to claim 1, wherein the mass spectrometry analysis involves tandem mass spectrometry (MS/MS) for identification and quantification of Borrelia-derived peptides.

3. The method according to claim 1, wherein the immunoassay is an enzyme-linked immunosorbent assay (ELISA) designed to detect peptide markers bound to the filamentous material.

4. The method according to claim 1, wherein the protein sequencing is conducted using high-throughput sequencing technologies for comprehensive identification of Borrelia strain peptide markers.

5. The method according to claim 1, further comprising comparing detected peptide markers with a reference database to confirm presence of one or more Borrelia strain indicative of Lyme disease.

6. The method according to claim 1, wherein the isolated composition is processed to remove non-specific proteins and impurities prior to analytical testing to enhance detection accuracy.

7. The method of claim 1, wherein a weight ratio (% W/W) between a total amount of affinity molecule attached to the filamentous material is about 0.5 to 2%.

8. The method of claim 1, wherein isolation of the said composition is contacting the filamentous material with a volume of a biological sample to allow the affinity molecule to capture one or more biomarkers present in the biological sample in a suitable condition, eluting captured biomarkers; and wherein the said method is a non-invasive process.

9. The method of claim 1, wherein the filamentous material is a non-imbibing material.

10. The method of claim 8, wherein the suitable condition comprises pH of the biological sample.

11. The method of claim 1, wherein the biological sample is not preserved in a refrigerated condition before contacting the filamentous material.

12. The method of claim 1, wherein the filamentous material is heated at a temperature about its glass transition temperature to allow its functionalization with one or more affinity molecule.

13. A diagnostic composition comprising: a biomarker derived from Borrelia strains; wherein the biomarker is bound or complexed to an affinity molecule; and the affinity molecule is bound or complexed to a filamentous material.

14. The diagnostic composition of claim 13, wherein the affinity molecule comprises salts and/or solvates.

15. The diagnostic composition of claim 13, wherein the composition is configured to detect a biomarker indicative of lyme disease.

16. The composition of claim 13, wherein the biomarker includes a protein, polypeptide, nucleic acids, lipids, antigens, carbohydrates, or glycanproteins, exososomes, extracellular vesicles unambiguously attributable to pathogenic Borrelia strains.

17. The composition of claim 13, wherein the affinity molecule is selected from the group consisting of dyes, metal ions, drugs, antibodies, recombinant antibodies, co-enzymes, vitamins, proteins, peptides, aptamers, receptor ligands, and lectins.

18. The diagnostic composition of claim 13, wherein the affinity molecule comprises a hydrogel.

19. The diagnostic composition of claim 13, wherein the filamentous material is selected from a natural or a synthetic fibre.

20. The diagnostic composition of claim 13, wherein the filamentous material comprises a polymer selected from nylon-6 and/or polyamide.
